(19) United States
(12) Patent Application Publication
(10) Pub. No.: US 2022/0323429 A1
(43) Pub. Date: Oct. 13, 2022

(54) PIMAVANSERIN FOR TREATING NEURODEGENERATIVE DISEASES

(71) Applicant: ACADIA Pharmaceuticals Inc., San Diego, CA (US)

(72) Inventors: Allan Bokser, San Diego, CA (US); Yvonne Adegbenle, San Marcos, CA (US); James Norton, Huntington Beach, CA (US)

(21) Appl. No.: 17/635,459

(22) PCT Filed: Aug. 13, 2020
(86) PCT No.: PCT/US2020/046212

§ 371 Date: Feb. 15, 2022

Related U.S. Application Data
(60) Provisional application No. 62/944,743, filed on Dec. 6, 2019.
(60) Provisional application No. 62/887,118, filed on Aug. 15, 2019.

(51) Int. Cl. A61K 31/4468; A61K 9/00; A61K 9/08

(52) U.S. Cl. CPC A61K 31/4468; A61K 9/0095; A61K 9/08; A61K 9/0053

(57) ABSTRACT

The disclosure provides, in part, a method of treating a disorder in a patient in need of pimavanserin, the method comprising administering to the patient about 2 mg to about 80 mg of pimavanserin via, e.g., an enteral feeding tube, once daily, wherein pimavanserin is dissolved in, mixed with, or sparkled onto, e.g., about 5 mL to about 150 mL of, a liquid vehicle or soft food vehicle at ambient or cold temperature before the administration.

### CROSS-REFERENCE TO RELATED APPLICATIONS

This application claims benefit of priority to U.S. Provisional Application No. 62/887,118 filed on Aug. 15, 2019, and U.S. Provisional Application No. 62/944,743 filed on Dec. 6, 2019, the entire contents of each of which are hereby incorporated by reference.

### BACKGROUND

Pimavanserin, is a selective serotonin 5-HT2A inverse agonist that has been approved by the U.S. Food and Drug Administration (FDA) for the treatment of hallucinations and delusions in patients with Parkinson's disease psychosis (PDP) as a solid oral capsule formulation containing 34 mg of pimavanserin. The PDP patient population typically comprises older adults who often have one or more chronic diseases in addition to Parkinson's disease and may be taking multiple medications. This population of older patients are inherently predisposed to swallowing disorders such as dysphagia, which could affect the proper administration of intact pimavanserin capsules or tablets (Pflug C et al., Dysphagia 2018, 33:41-50; Kalf J G et al., Parkinsonism Relat. Disord. 2012, 18:311-5).

Dysphagia or difficulty swallowing is a common clinical problem estimated to occur in up to 40% of the adult population (Fields J et al., Curr. Ther. Res. Clin. Exp. 2015, 77:79-82; Schiele, J T et al., Eur. J. Clin. Pharmacol. 2013, 69:937-48). Dysphagia is particularly common among the elderly population and in patients prescribed oral dosage forms for treating chronic diseases such as Parkinson's disease, stroke, Alzheimer's disease, dementia, and other neurodegenerative conditions. For instance, it has been reported that up to 80% or more of Parkinson's disease patients experience difficulty swallowing solid dosage forms configured for oral administration (Pflug C et al., Dysphagia 2018, 33:41-50; Kalf J G et al., Parkinsonism Relat. Disord. 2012, 18:311-5).

Small amounts of liquids and/or soft foods can be used as a suitable vehicle(s) for administration in the absence of a suitable dosage formulation appropriate for the targeted patient population, e.g., older patients with swallowing difficulties. However, altering the original solid dosage form may affect the absorption, stability, and delivery of the drug, and can lead to significant changes in the pharmacological effect. Therefore, the selection of a liquid or soft food vehicle becomes an important aspect for administering the drug product appropriately to the patients with swallowing difficulties.

### SUMMARY

The disclosure provides, in part, a method of treating a disorder (e.g., a psychosis secondary to a neurodegenerative disorder, schizophrenia, or major depressive disorder) in a patient in need of pimavanserin, the method comprising administering to the patient about 2 mg to about 80 mg of pimavanserin (e.g., via an enteral feeding tube) once daily, wherein pimavanserin is dissolved in or mixed with about 5 mL to about 150 mL of a liquid vehicle at ambient or cold temperature before the administration.

In another aspect, provided herein is a method of treating a disorder in a patient in need of pimavanserin, the method comprising administering to the patient about 2 mg to about 80 mg of pimavanserin via an enteral feeding tube once daily, wherein pimavanserin is dissolved in or mixed with a liquid vehicle at ambient or cold temperature before the administration, wherein the solution or mixture is stable in the nasogastric tube at ambient or cold temperature.

Provided herein, in part, is a method of treating a disorder in a patient in need of pimavanserin, the method comprising administering to the patient about 2 mg to about 80 mg of pimavanserin once daily, wherein the administration comprises the steps of:

a) opening a pharmaceutically acceptable capsule comprising pimavanserin;

b) mixing the contents of the capsule with about 5 mL to about 150 mL (e.g., about 5 mL to about 60 mL) of a liquid vehicle or sprinkling the contents of the capsule onto about 5 mL to about 150 mL of a soft food vehicle at ambient or cold temperature; and

c) delivering to the patient, optionally via an enteral feeding tube the contents of the capsule with the liquid vehicle to the patient, or orally delivering the contents of the capsule with the soft food vehicle to the patient.

Also provided herein is a method of treating a disorder in a patient in need of pimavanserin, the method comprising administering to the patient about 2 mg to about 80 mg of pimavanserin once daily, wherein the administration comprises the steps of:

a) opening a pharmaceutically acceptable capsule comprising pimavanserin;

b) mixing the contents of the capsule with about 5 mL to about 150 mL of a liquid vehicle or sprinkling the contents of the capsule onto about 5 mL to about 150 mL of a soft food vehicle at ambient or cold temperature; and

c) orally delivering the contents of the capsule with the soft food vehicle to the patient.

Provided herein, in part is a method of treating a disorder in a patient in need of pimavanserin, the method comprising administering to the patient about 2 mg to about 80 mg of pimavanserin once daily, wherein the administration comprises the steps of:

a) crushing a pharmaceutically acceptable tablet comprising pimavanserin;

b) mixing the crushed tablet with about 5 mL to about 150 mL of a liquid vehicle or sprinkling the crushed tablet onto about 5 mL to about 150 mL of a soft food vehicle at ambient or cold temperature; and

c) delivering to the patient, optionally via an enteral feeding tube, the crushed tablet with the liquid vehicle to the patient, or orally delivering the crushed tablet with the soft food vehicle to the patient.

Also provided herein is a method of treating a disorder in a patient in need of pimavanserin, the method comprising administering to the patient about 2 mg to about 80 mg of pimavanserin once daily, wherein the administration comprises the steps of:

a) crushing a pharmaceutically acceptable tablet comprising pimavanserin;

b) mixing the crushed tablet with about 5 mL to about 150 mL of a liquid vehicle or sprinkling the crushed tablet onto about 5 mL to about 150 mL of a soft food vehicle at ambient or cold temperature; and

c) orally delivering the crushed tablet with the soft food vehicle to the patient.

Also provided herein is a method of treating a disorder in a patient in need thereof, the method comprising administering to the patient about 2 mg to about 80 mg of pimavanserin via a feeding tube once daily, wherein pimavanserin is dissolved in or mixed with about 5 mL to about 150 mL of a liquid vehicle before the administration. In some embodiments, the feeding tube is an oral feeding tube. In certain embodiments, the feeding tube is an enteral feeding tube.

In other embodiments, about 2 mg to about 80 mg of pimavanserin is a unit dose comprising pimavanserin tartrate. The contemplated psychosis may include Parkinson's disease psychosis or Alzheimer's disease psychosis.

The contemplated methods may comprise administering to the patient about 34 mg of pimavanserin. In certain embodiments, the methods may comprise administering to the patient about 20 mg of pimavanserin. In some embodiments, the methods may comprise administering to the patient about 17 mg of pimavanserin. In other embodiments, the methods may comprise administering to the patient about 10 mg of pimavanserin.

In certain embodiments of the methods disclosed herein, pimavanserin may be dissolved in or mixed with about 5 mL to about 60 mL of the liquid vehicle.

The enteral feeding tube of the methods described herein may be a nasogastric tube, orogastric tube, nasoenteric tube, or oroenteric tube.

In certain embodiments the enteral feeding tube of the methods described herein may be a nasogastric tube.

In one aspect, the present disclosure provides a method of treating a disorder in a patient in need of pimavanserin, the method comprising administering to the patient 34 mg of pimavanserin once daily, wherein pimavanserin is dissolved in or mixed with about 5 mL to about 150 mL of a liquid vehicle at ambient or cold temperature before the administration.

In another aspect, provided herein is a method of treating a disorder in a patient in need of pimavanserin, the method comprising administering to the patient 10 mg of pimavanserin once daily, wherein pimavanserin is dissolved in or mixed with about 5 mL to about 150 mL of a liquid vehicle at ambient or cold temperature before the administration, wherein the patient is being treated concurrently with a strong CYP3A4 inhibitor.

Contemplated methods include a method of treating a disorder in a patient in need of pimavanserin, the method comprising administering to the patient 34 mg of pimavanserin via an enteral feeding tube once daily, wherein pimavanserin is dissolved in or mixed with a liquid vehicle at ambient or cold temperature before the administration, wherein the solution or mixture is stable in the enteral feeding tube at ambient or cold temperature.

Also provided herein is a method of treating a disorder in a patient in need of pimavanserin, the method comprising administering to the patient 10 mg of pimavanserin via an enteral feeding tube once daily, wherein pimavanserin is dissolved in or mixed with a liquid vehicle at ambient or cold temperature before the administration, wherein the solution or mixture is stable in the enteral feeding tube at ambient or cold temperature, wherein the patient is being treated concurrently with a strong CYP3A4 inhibitor.

The present disclosure provides, in part, a method of treating a disorder in a patient in need of pimavanserin, the method comprising administering to the patient 34 mg of pimavanserin once daily, wherein the administration comprises the steps of:

a) opening a pharmaceutically acceptable capsule comprising pimavanserin;

b) mixing the contents of the capsule with about 5 mL to about 150 mL of a liquid vehicle or sprinkling the contents of the capsule onto about 5 mL to about 150 mL of a soft food vehicle at ambient or cold temperature; and

c) delivering to the patient, optionally via an enteral feeding tube, the contents of the capsule with the liquid vehicle to the patient, or orally delivering the contents of the capsule with the soft food vehicle to the patient.

Provided methods include a method of treating a disorder in a patient in need of pimavanserin, the method comprising administering to the patient 34 mg of pimavanserin once daily, wherein the administration comprises the steps of:

a) opening a pharmaceutically acceptable capsule comprising pimavanserin;

b) mixing the contents of the capsule with about 5 mL to about 150 mL of a liquid vehicle or sprinkling the contents of the capsule onto about 5 mL to about 150 mL of a soft food vehicle at ambient or cold temperature; and

c) orally delivering the contents of the capsule with the soft food vehicle to the patient.

Provided herein, in part, is a method of treating a disorder in a patient in need of pimavanserin, the method comprising administering to the patient about 10 mg of pimavanserin once daily, wherein the administration comprises the steps of:

a) crushing a pharmaceutically acceptable tablet comprising pimavanserin;

b) mixing the crushed tablet with about 5 mL to about 150 mL of a liquid vehicle or sprinkling the crushed tablet onto about 5 mL to about 150 mL of a soft food vehicle at ambient or cold temperature; and

c) delivering to the patient, optionally via an enteral feeding tube, the crushed tablet with the liquid vehicle to the patient, or orally delivering the crushed tablet with the soft food vehicle to the patient;

wherein the patient is being treated concurrently with a strong CYP3A4 inhibitor.

In one aspect, a method of treating a disorder in a patient in need of pimavanserin, the method comprising administering to the patient about 10 mg of pimavanserin once daily, wherein the administration comprises the steps of:

a) crushing a pharmaceutically acceptable tablet comprising pimavanserin;

b) mixing the crushed tablet with about 5 mL to about 150 mL of a liquid vehicle or sprinkling the crushed tablet onto about 5 mL to about 150 mL of a soft food vehicle at ambient or cold temperature; and

c) orally delivering the crushed tablet with the soft food vehicle to the patient;

wherein the patient is being treated concurrently with a strong CYP3A4 inhibitor, is provided.

Also provided herein is a method of treating hallucinations and delusions associated with dementia in a patient in need thereof, comprising administering to the patient a pimavanserin composition via a nasogastric tube once daily, wherein the pimavanserin composition comprises about 34 mg pimavanserin and about 5 mL to about 60 mL of a liquid vehicle.

The present disclosure provides, in part, a method of treating hallucinations and delusions associated with dementia related to psychosis in a patient in need thereof, comprising administering to the patient a pimavanserin composition via a nasogastric tube once daily, wherein the pimavanserin composition comprises about 34 mg pimavanserin and about 5 mL to about 60 mL of a liquid vehicle.

In another aspect, the disclosure provides a method of treating hallucinations and delusions associated with Parkinson's disease in a patient in need thereof, comprising administering to the patient a pimavanserin composition via a nasogastric tube once daily, wherein the pimavanserin composition comprises about 34 mg pimavanserin and about 5 mL to about 60 mL of a liquid vehicle.

Provided in the present disclosure includes a method of treating hallucinations and delusions associated with Parkinson's disease in a patient in need thereof, the method comprising administering to the patient a pimavanserin composition via a nasogastric tube once daily, wherein the pimavanserin composition comprises about 34 mg pimavanserin and a liquid vehicle, wherein the composition is stable in the nasogastric tube at ambient or cold temperature.

Also provided herein is a method of treating hallucinations and delusions associated with Parkinson's disease in a patient in need thereof, comprising administering to the patient about 34 mg pimavanserin once daily, wherein the administration comprises the steps of:

a) opening a pharmaceutically acceptable capsule comprising pimavanserin;

b) mixing the contents of the capsule with about 5 mL to about 60 mL of a liquid vehicle or sprinkling the contents of the capsule onto about 5 mL to about 60 mL of a soft food vehicle at ambient or cold temperature; and

c) delivering via a nasogastric tube the contents of the capsule with the liquid vehicle to the patient, or orally delivering the contents of the capsule with the soft food vehicle to the patient.

The present disclosure provides, in part, a method of treating hallucinations and delusions associated with Parkinson's disease in a patient in need thereof, the method comprising administering to the patient a pimavanserin composition via an oral or enteral feeding tube once daily, wherein the pimavanserin composition comprises about 34 mg pimavanserin and about 5 mL to about 60 mL of a liquid vehicle.

In one aspect, the present disclosure provides a method of treating hallucinations and delusions associated with Parkinson's disease in a patient in need thereof, comprising administering to the patient a pimavanserin composition via a nasogastric tube once daily, wherein the pimavanserin composition comprises about 10 mg pimavanserin and about 5 mL to about 60 mL of a liquid vehicle, wherein the patient is being treated concurrently with a strong CYP3A4 inhibitor.

Provided herein, in part, is a method of treating hallucinations and delusions associated with dementia in a patient in need thereof, comprising administering to the patient a pimavanserin composition via a nasogastric tube once daily, wherein the pimavanserin composition comprises about 10 mg pimavanserin and about 5 mL to about 60 mL of a liquid vehicle, wherein the patient is being treated concurrently with a strong CYP3A4 inhibitor.

Also provided here in is a method of treating hallucinations and delusions associated with dementia related to psychosis in a patient in need thereof, comprising administering to the patient a pimavanserin composition via a nasogastric tube once daily, wherein the pimavanserin composition comprises about 10 mg pimavanserin and about 5 mL to about 60 mL of a liquid vehicle, wherein the patient is being treated concurrently with a strong CYP3A4 inhibitor.

In another aspect, a method of treating hallucinations and delusions associated with Parkinson's disease in a patient in need thereof, the method comprising administering to the patient a pimavanserin composition via a nasogastric tube once daily, wherein the pimavanserin composition comprises about 10 mg pimavanserin and a liquid vehicle, wherein the composition is stable in the nasogastric tube at ambient or cold temperature, wherein the patient is being treated concurrently with a strong CYP3A4 inhibitor is provided.

Contemplated methods include a method of treating hallucinations and delusions associated with Parkinson's disease in a patient in need thereof, comprising administering to the patient about 10 mg pimavanserin once daily, wherein the administration comprises the steps of:

a) opening a pharmaceutically acceptable capsule comprising pimavanserin;

b) mixing the contents of the capsule with about 5 mL to about 60 mL of a liquid vehicle or sprinkling the contents of the capsule onto about 5 mL to about 60 mL of a soft food vehicle at ambient or cold temperature; and

c) delivering via a nasogastric tube the contents of the capsule with the liquid vehicle to the patient, or orally delivering the contents of the capsule with the soft food vehicle to the patient;

wherein the patient is being treated concurrently with a strong CYP3A4 inhibitor.

The present disclosure provides, in part, a method of treating hallucinations and delusions associated with Parkinson's disease in a patient in need thereof, the method comprising administering to the patient a pimavanserin composition via an oral or enteral feeding tube once daily, wherein the pimavanserin composition comprises about 10 mg pimavanserin and about 5 mL to about 60 mL of a liquid vehicle, wherein the patient is being treated concurrently with a strong CYP3A4 inhibitor.

The contemplated patient may have swallowing difficulties. In some embodiments, the pimavanserin is in the form of the tartrate salt.

### BRIEF DESCRIPTION OF THE DRAWINGS

FIG. 1 shows the schematic of study design as described in Example 3.

FIG. 2 shows the testing scheme for each food vehicle mixed with pimavanserin as described in Example 3.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder and the like.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

As used herein, a “diluent,” “bulking agent,” and “filler” refer to an ingredient (excipient) in a pharmaceutical composition that lacks pharmacological activity but may be pharmaceutically necessary or desirable, e.g. to enhance or improve the properties of the pharmaceutical blend for manufacturing or physiological purposes. For example, a diluent or filler may be used to increase the bulk of a potent drug whose mass is too small for manufacture or administration.

The term “pharmaceutical composition” as used herein refers to a composition comprising at least one compound as disclosed herein formulated together with one or more pharmaceutically acceptable carriers.

In the present specification, the term “therapeutically effective amount” means the amount of the subject compound that will elicit the biological or medical response of a tissue, system, animal or human that is being sought by the researcher, veterinarian, medical doctor or other clinician. The compounds of the disclosure are administered in therapeutically effective amounts to treat a disease. Alternatively, a therapeutically effective amount of a compound is the quantity required to achieve a desired therapeutic and/or prophylactic effect, such as an amount which results in the treatment of depression.

The term “encapsulation” refers to a range of techniques used to enclose medicines in a shell, e.g. a two-piece capsule, such as a two-piece hard shell capsule. The capsule referred to herein may be taken orally. Capsules may be designed with a telescoping cap and body manufactured from e.g. gelatin or cellulose.

An “agonist” is defined as a compound that increases the basal activity of a receptor (e.g. signal transduction mediated by the receptor).

As used herein, an “inverse agonist” is defined as a compound, which reduces, or suppresses the basal activity of a receptor, such that the compound is not technically an antagonist but, rather, is an agonist with negative intrinsic activity.

As used herein, “antagonist” refers to a compound that binds to a receptor to form a complex that does not give rise to any response, as if the receptor was unoccupied. An antagonist attenuates the action of an agonist on a receptor. An antagonist may bind reversibly or irreversibly, effectively eliminating the activity of the receptor permanently or at least until the antagonist is metabolized or dissociates or is otherwise removed by a physical or biological process.

As used herein an “intact” drug product refers to solid oral dosage forms such as granules, pellets, powders, as well as certain specific modified release drug products such as capsules or packets containing beads that are labeled to be administered via sprinkling.

The term “sprinkled” or “sprinkling” as used herein refers to emptying the contents of a capsule comprising pimavanserin onto a liquid or soft food vehicle before oral or enteric administration.

The term “Stability Assay” as used herein refers to the HPLC assay as described in Example 1 of the present disclosure.

The term “Nasogastric Tube Compatibility Study” refers to the HPLC assay as described in Example 2 of the present disclosure.

“Disease,” “disorder,” and “condition” are used interchangeably herein.

The term “nasogastric tube” as used herein is an enteral feeding tube that starts in the nose and ends in the stomach.

The term “orogastric tube” as used herein in an enteral feeding tube that starts in the mouth and ends in the stomach.

The term “nasoenteric tube” as used herein is an enteral feeding tube that starts in the nose and ends in the intestines. Subtypes of a nasoenteric tube includes nasojejunal and nasoduodenal tubes.

The term “oroenteric tube” as used herein is an enteral feeding tube that starts in the mouth and ends in the intestines.

The term “gastrostomy tube” as used herein is an enteral feeding tube that is placed through the skin of the abdomen straight to the stomach. Subtypes of a gastrostomy tube include PEG, PRG, and button tubes.

The term “jejunostomy tube” as used herein is an enteral feeding tube that is placed through the skin of the abdomen straight into the intestines. Subtypes of a jejunostomy tube includes PEJ and PRJ tubes.

### Methods

Provided herein, in part, is a method of treating a disease (e.g., psychosis secondary to a neurodegenerative disorder, schizophrenia, or major depressive disorder) in a patient in need of pimavanserin, the method comprising administering to the patient about 34 mg of pimavanserin once daily, wherein pimavanserin is dissolved in or mixed with a liquid vehicle or sprinkled onto a soft food vehicle before the administration.

In one aspect, the disclosure provides a method of treating a disorder in a patient in need of pimavanserin, the method comprising administering to the patient about 2 mg to about 80 mg of pimavanserin once daily, wherein pimavanserin is dissolved in or mixed with about 5 mL to about 150 mL of a liquid vehicle at ambient or cold temperature before the administration.

Also provided herein is a method of treating a disease in a patient in need of pimavanserin, the method comprising administering to the patient about 2 mg to about 80 mg of pimavanserin via an enteral feeding tube once daily, wherein pimavanserin is dissolved in or mixed with a liquid vehicle at ambient or cold temperature before the administration, wherein the solution or mixture is stable in the nasogastric tube at ambient or cold temperature.

For example, disclosed herein is a method of treating a disorder in a patient in need of pimavanserin, the method comprising administering to the patient about 2 mg to about 80 mg of pimavanserin once daily, wherein the administration comprises the steps of:

a) opening a pharmaceutically acceptable capsule comprising pimavanserin;

b) mixing the contents of the capsule with about 5 mL to about 150 mL of a liquid vehicle or sprinkling the contents of the capsule onto about 5 mL to about 150 mL of a soft food vehicle at ambient or cold temperature; and

c) delivering to the patient, optionally via an enteral feeding tube the contents of the capsule with the liquid vehicle to the patient, or orally delivering the contents of the capsule with the soft food vehicle to the patient.

Contemplated methods include a method of treating a disorder in a patient in need of pimavanserin, the method comprising administering to the patient about 2 mg to about 80 mg of pimavanserin once daily, wherein the administration comprises the steps of:

a) opening a pharmaceutically acceptable capsule comprising pimavanserin;

b) mixing the contents of the capsule with about 5 mL to about 150 mL of a liquid vehicle or sprinkling the contents of the capsule onto about 5 mL to about 150 mL of a soft food vehicle at ambient or cold temperature; and

c) orally delivering the contents of the capsule with the soft food vehicle to the patient.

Provided herein, in part is a method of treating a disorder in a patient in need of pimavanserin, the method comprising administering to the patient about 2 mg to about 80 mg of pimavanserin once daily, wherein the administration comprises the steps of:
a) crushing a pharmaceutically acceptable tablet comprising pimavanserin;
b) mixing the crushed tablet with about 5 mL to about 150 mL of a liquid vehicle or sprinkling the crushed tablet onto about 5 mL to about 150 mL of a soft food vehicle at ambient or cold temperature; and
c) delivering to the patient, optionally via an enteral feeding tube, the crushed tablet with the liquid vehicle to the patient, or orally delivering the crushed tablet with the soft food vehicle to the patient.

Also provided herein is a method of treating a disorder in a patient in need of pimavanserin, the method comprising administering to the patient about 2 mg to about 80 mg of pimavanserin once daily, wherein the administration comprises the steps of:
a) crushing a pharmaceutically acceptable tablet comprising pimavanserin;
b) mixing the crushed tablet with about 5 mL to about 150 mL of a liquid vehicle or sprinkling the crushed tablet onto about 5 mL to about 150 mL of a soft food vehicle at ambient or cold temperature; and
c) orally delivering the crushed tablet with the soft food vehicle to the patient.

In other embodiments of a method described herein, the contents of the capsule with the soft vehicle is orally delivered to the patient via an enteral feeding tube.

In some embodiments of a method described herein, the disorder is a psychosis secondary to a neurodegenerative disorder (e.g., wherein the disorder is a psychosis secondary to Parkinson's disease, Alzheimer's disease, vascular dementia, or Lewy body dementia). In other embodiments, the disorder is schizophrenia or major depressive disorder. In other embodiments, the disorder is a negative symptom of schizophrenia.

The contemplated methods of the present disclosure may comprise administering to the patient about 5 mg to about 45 mg of pimavanserin. For example, the methods may comprise administering to the patient about 34 mg of pimavanserin. In certain embodiments, the methods may comprise administering to the patient about 20 mg of pimavanserin. In some embodiments, the methods may comprise administering to the patient about 17 mg of pimavanserin. In other embodiments, the methods may comprise administering to the patient about 10 mg of pimavanserin, wherein the patient is being treated concurrently with a strong CYP3A4 inhibitor.

In certain embodiments, pimavanserin is administered to the patient via an enteral feeding tube.

The enteral feeding tube of a method described herein may a nasogastric tube. In certain embodiments, the enteral feeding tube may be an orogastric tube. In some embodiments, the enteral feeding tube may be a nasoenteric tube. In other embodiments, the enteral feeding tube may be an oroenteric tube. In certain embodiments, the enteral feeding tube may be a gastrostomy tube. In some embodiments, the enteral feeding tube may be a jejunostomy tube.

In certain embodiments of the methods disclosed herein, pimavanserin may be dissolved in or mixed with about 5 mL to about 100 mL of the liquid vehicle. For example, pimavanserin may be dissolved in or mixed with about 5 mL to about 60 mL of the liquid vehicle. In certain embodiments, pimavanserin may be dissolved in or mixed with about 5 mL to about 30 mL, about 5 mL to about 15 mL, about 10 mL to about 80 mL, about 10 mL to about 50 mL, about 10 mL to about 20 mL, about 15 mL to about 70 mL, about 15 mL to about 45 mL, about 15 mL to about 30 mL, about 20 mL to about 80 mL, about 20 mL to about 60 mL, about 20 mL to about 40 mL, about 30 mL to about 80 mL, about 30 mL to about 60 mL, or about 30 mL to about 45 mL of the liquid vehicle.

In some embodiments, pimavanserin may be dissolved in or mixed with about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 80 mL, about 85 mL, about 90 mL, about 95 mL, or about 100 mL of the liquid vehicle. In certain embodiments, pimavanserin may be dissolved in or mixed with about 15 mL of the liquid vehicle. In certain embodiments, pimavanserin may be dissolved in or mixed with about 30 mL of the liquid vehicle.

In one aspect, the present disclosure provides a method of treating a disorder in a patient in need of pimavanserin, the method comprising administering to the patient 34 mg of pimavanserin once daily, wherein pimavanserin is dissolved in or mixed with about 5 mL to about 150 mL of a liquid vehicle at ambient or cold temperature before the administration.

In another aspect, provided herein is a method of treating a disorder in a patient in need of pimavanserin, the method comprising administering to the patient 10 mg of pimavanserin once daily, wherein pimavanserin is dissolved in or mixed with about 5 mL to about 150 mL of a liquid vehicle at ambient or cold temperature before the administration, wherein the patient is being treated concurrently with a strong CYP3A4 inhibitor.

Contemplated methods include a method of treating a disorder in a patient in need of pimavanserin, the method comprising administering to the patient 34 mg of pimavanserin via an enteral feeding tube once daily, wherein pimavanserin is dissolved in or mixed with a liquid vehicle at ambient or cold temperature before the administration, wherein the solution or mixture is stable in the enteral feeding tube at ambient or cold temperature.

Also provided herein is a method of treating a disorder in a patient in need of pimavanserin, the method comprising administering to the patient 10 mg of pimavanserin via an enteral feeding tube once daily, wherein pimavanserin is dissolved in or mixed with a liquid vehicle at ambient or cold temperature before the administration, wherein the solution or mixture is stable in the enteral feeding tube at ambient or cold temperature, wherein the patient is being treated concurrently with a strong CYP3A4 inhibitor.

The present disclosure provides, in part, a method of treating a disorder in a patient in need of pimavanserin, the method comprising administering to the patient 34 mg of pimavanserin once daily, wherein the administration comprises the steps of:
a) opening a pharmaceutically acceptable capsule comprising pimavanserin;
b) mixing the contents of the capsule with about 5 mL to about 150 mL of a liquid vehicle or sprinkling the contents of the capsule onto about 5 mL to about 150 mL of a soft food vehicle at ambient or cold temperature; and
c) delivering to the patient, optionally via an enteral feeding tube, the contents of the capsule with the liquid vehicle to the patient, or orally delivering the contents of the capsule with the soft food vehicle to the patient.

Provided methods include a method of treating a disorder in a patient in need of pimavanserin, the method comprising administering to the patient 34 mg of pimavanserin once daily, wherein the administration comprises the steps of:
a) opening a pharmaceutically acceptable capsule comprising pimavanserin;
b) mixing the contents of the capsule with about 5 mL to about 150 mL of a liquid vehicle or sprinkling the contents of the capsule onto about 5 mL to about 150 mL of a soft food vehicle at ambient or cold temperature; and
c) orally delivering the contents of the capsule with the soft food vehicle to the patient.

Provided herein, in part, is a method of treating a disorder in a patient in need of pimavanserin, the method comprising administering to the patient about 10 mg of pimavanserin once daily, wherein the administration comprises the steps of:
a) crushing a pharmaceutically acceptable tablet comprising pimavanserin;
b) mixing the crushed tablet with about 5 mL to about 150 mL of a liquid vehicle or sprinkling the crushed tablet onto about 5 mL to about 150 mL of a soft food vehicle at ambient or cold temperature; and
c) delivering to the patient, optionally via an enteral feeding tube, the crushed tablet with the liquid vehicle to the patient, or orally delivering the crushed tablet with the soft food vehicle to the patient;
   wherein the patient is being treated concurrently with a strong CYP3A4 inhibitor.

In one aspect, a method of treating a disorder in a patient in need of pimavanserin, the method comprising administering to the patient about 10 mg of pimavanserin once daily, wherein the administration comprises the steps of:
a) crushing a pharmaceutically acceptable tablet comprising pimavanserin;
b) mixing the crushed tablet with about 5 mL to about 150 mL of a liquid vehicle or sprinkling the crushed tablet onto about 5 mL to about 150 mL of a soft food vehicle at ambient or cold temperature; and
c) orally delivering the crushed tablet with the soft food vehicle to the patient;
   wherein the patient is being treated concurrently with a strong CYP3A4 inhibitor, is provided.

The present disclosure also provides a method of treating hallucinations and delusions associated with Parkinson's disease in a patient in need thereof, comprising administering to the patient a pimavanserin composition via a nasogastric tube once daily, wherein the pimavanserin composition comprises about 34 mg pimavanserin and about 5 mL to about 60 mL of a liquid vehicle.

Also provided herein is a method of treating hallucinations and delusions associated with dementia in a patient in need thereof, comprising administering to the patient a pimavanserin composition via a nasogastric tube once daily, wherein the pimavanserin composition comprises about 34 mg pimavanserin and about 5 mL to about 60 mL of a liquid vehicle.

The present disclosure provides, in part, a method of treating hallucinations and delusions associated with dementia related to psychosis in a patient in need thereof, comprising administering to the patient a pimavanserin composition via a nasogastric tube once daily, wherein the pimavanserin composition comprises about 34 mg pimavanserin and about 5 mL to about 60 mL of a liquid vehicle.

Also provided herein is a method of treating hallucinations and delusions associated with Parkinson's disease in a patient in need thereof, the method comprising administering to the patient a pimavanserin composition via a nasogastric tube once daily, wherein the pimavanserin composition comprises about 34 mg pimavanserin and a liquid vehicle, wherein the composition is stable in the nasogastric tube at ambient or cold temperature.

For example, the present disclosure provides a method of treating hallucinations and delusions associated with Parkinson's disease in a patient in need thereof, comprising administering to the patient about 34 mg pimavanserin once daily, wherein the administration comprises the steps of:
a) opening a pharmaceutically acceptable capsule comprising pimavanserin;
b) mixing the contents of the capsule with about 5 mL to about 60 mL of a liquid vehicle or sprinkling the contents of the capsule onto about 5 mL to about 60 mL of a soft food vehicle at ambient or cold temperature; and
c) delivering via a nasogastric tube the contents of the capsule with the liquid vehicle to the patient, or orally delivering the contents of the capsule with the soft food vehicle to the patient.

In one aspect, the present disclosure provides a method of treating hallucinations and delusions associated with Parkinson's disease in a patient in need thereof, comprising administering to the patient a pimavanserin composition via a nasogastric tube once daily, wherein the pimavanserin composition comprises about 10 mg pimavanserin and about 5 mL to about 60 mL of a liquid vehicle, wherein the patient is being treated concurrently with a strong CYP3A4 inhibitor.

Provided herein, in part, is a method of treating hallucinations and delusions associated with dementia in a patient in need thereof, comprising administering to the patient a pimavanserin composition via a nasogastric tube once daily, wherein the pimavanserin composition comprises about 10 mg pimavanserin and about 5 mL to about 60 mL of a liquid vehicle, wherein the patient is being treated concurrently with a strong CYP3A4 inhibitor.

Also provided here in is a method of treating hallucinations and delusions associated with dementia related to psychosis in a patient in need thereof, comprising administering to the patient a pimavanserin composition via a nasogastric tube once daily, wherein the pimavanserin composition comprises about 10 mg pimavanserin and about 5 mL to about 60 mL of a liquid vehicle, wherein the patient is being treated concurrently with a strong CYP3A4 inhibitor.

In another aspect, a method of treating hallucinations and delusions associated with Parkinson's disease in a patient in need thereof, the method comprising administering to the patient a pimavanserin composition via a nasogastric tube once daily, wherein the pimavanserin composition comprises about 10 mg pimavanserin and a liquid vehicle, wherein the composition is stable in the nasogastric tube at ambient or cold temperature, wherein the patient is being treated concurrently with a strong CYP3A4 inhibitor is provided.

Contemplated methods include a method of treating hallucinations and delusions associated with Parkinson's disease in a patient in need thereof, comprising administering to the patient about 10 mg pimavanserin once daily, wherein the administration comprises the steps of:
a) opening a pharmaceutically acceptable capsule comprising pimavanserin;
b) mixing the contents of the capsule with about 5 mL to about 60 mL of a liquid vehicle or sprinkling the contents of the capsule onto about 5 mL to about 60 mL of a soft food vehicle at ambient or cold temperature; and
c) delivering via a nasogastric tube the contents of the capsule with the liquid vehicle to the patient, or orally delivering the contents of the capsule with the soft food vehicle to the patient;
   wherein the patient is being treated concurrently with a strong CYP3A4 inhibitor.

The present disclosure provides, in part, a method of treating hallucinations and delusions associated with Parkinson's disease in a patient in need thereof, the method comprising administering to the patient a pimavanserin composition via an oral or enteral feeding tube once daily, wherein the pimavanserin composition comprises about 10 mg pimavanserin and about 5 mL to about 60 mL of a liquid vehicle, wherein the patient is being treated concurrently with a strong CYP3A4 inhibitor.

In some embodiments of the disclosed methods, pimavanserin is dissolved in or mixed with about 5 mL to about 15 mL of the liquid vehicle. For example, pimavanserin is dissolved in or mixed with about 15 mL of the liquid vehicle. In other embodiments, pimavanserin is dissolved in or mixed with about 30 mL of the liquid vehicle.

In certain embodiments of the disclosed methods, the contents of the capsule comprising pimavanserin are mixed with about 5 mL to about 15 mL of the liquid vehicle or the contents of the capsule comprising pimavanserin are sprinkled onto about 5 mL to about 15 mL of the soft food vehicle.

In other embodiments, pimavanserin or the contents of the capsule are dissolved in or mixed with 1 mL to 250 mL, or 5 mL to 200 mL, or 5 mL to 150 mL, or 5 mL to 100 mL, or 5 mL to 75 mL, or 5 mL to 50 mL, or 5 mL to 40 mL, or 5 mL to 30 mL, or 5 mL to 20, or 5 mL to 10 mL of the liquid vehicle at ambient or cold temperatures before the administration. In some embodiments, pimavanserin or the contents of the capsule are dissolved in or mixed with about 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 22 mL, 23 mL, 24 mL, 25 mL, 26 mL, 27 mL, 28 mL, 29 mL, 30 mL, 31 mL, 32 mL, 33 mL, 34 mL, 35 mL, 36 mL, 37 mL, 38 mL, 39 mL, 40 mL, 41 mL 42 mL, 43 mL, 44 mL, 45 mL, 46 mL, 47 mL, 48 mL, 49 mL, 50 mL, 51 mL, 52 mL, 53 mL, 54 mL, 55 mL, 58 mL, 57 mL, 58 mL, 59 mL, 60 mL, 61 mL, 62 mL, 63 mL, 64 mL, 65 mL, 66 mL, 67 mL, 68 mL, 69 mL, 70 mL, 71 mL, 72 mL, 73 mL, 74 mL, 75 mL, 76 mL, 77 mL, 78 mL, 79 mL 80 mL, 81 mL, 82 mL, 83 mL, 84 mL, 85 mL, 86 mL, 87 mL, 88 mL, 89 mL, 90 mL, 91 mL, 92 mL, 93 mL, 94 mL, 95 mL, 96 mL, 97 mL, 98 mL, 99 mL, or 100 mL of the liquid vehicle at ambient or cold temperature before the administration.

In certain embodiments, the contents of the pharmaceutically acceptable capsule comprising pimavanserin are sprinkled onto 1 mL to 250 mL, or 5 mL to 200 mL, or 5 mL to 150 mL, or 5 mL to 100 mL, or 5 mL to 75 mL, or 5 mL to 50 mL, or 5 mL to 40 mL, or 5 mL to 30 mL, or 5 mL to 20, or 5 mL to 10 mL of the soft food vehicle at ambient or cold temperatures before the administration. In some embodiments, the contents of the pharmaceutically acceptable capsule comprising pimavanserin are sprinkled onto about 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 22 mL, 23 mL, 24 mL, 25 mL, 26 mL, 27 mL, 28 mL, 29 mL, 30 mL, 31 mL, 32 mL, 33 mL, 34 mL, 35 mL, 36 mL, 37 mL, 38 mL, 39 mL, 40 mL, 41 mL 42 mL, 43 mL, 44 mL, 45 mL, 46 mL, 47 mL, 48 mL, 49 mL, 50 mL, 51 mL, 52 mL, 53 mL, 54 mL, 55 mL, 58 mL, 57 mL, 58 mL, 59 mL, 60 mL, 61 mL, 62 mL, 63 mL, 64 mL, 65 mL, 66 mL, 67 mL, 68 mL, 69 mL, 70 mL, 71 mL, 72 mL, 73 mL, 74 mL, 75 mL, 76 mL, 77 mL, 78 mL, 79 mL 80 mL, 81 mL, 82 mL, 83 mL, 84 mL, 85 mL, 86 mL, 87 mL, 88 mL, 89 mL, 90 mL, 91 mL, 92 mL, 93 mL, 94 mL, 95 mL, 96 mL, 97 mL, 98 mL, 99 mL, or 100 mL of the soft food vehicle at ambient or cold temperature before the administration.

In some embodiments, the disclosed methods provide a method of administering to the patient about 34 mg of pimavanserin once daily; wherein the administration comprises the steps of: a) opening a pharmaceutically acceptable capsule comprising pimavanserin; b) mixing the contents of the capsule with about 5 mL to about 60 mL of a liquid vehicle; and c) delivering via a nasogastric tube the contents of the capsule with the liquid vehicle to the patient. In some embodiments, pimavanserin is dissolved in or mixed with about 5 mL to about 60 mL of a liquid vehicle at ambient or cold temperature before the administration. In some embodiments, pimavanserin is dissolved in or mixed with about 5 mL to about 15 mL of the liquid vehicle at ambient or cold temperature before the administration.

In certain embodiments of a method disclosed herein, the nasogastric tube is rinsed once with an additional 5 to 30 mL of the liquid vehicle. In an embodiment, the nasogastric tube is rinsed twice with 5 to 15 mL of the liquid vehicle. For example, the nasogastric tube is rinsed with an additional 5 mL to 20 mL of the liquid vehicle. For example, the nasogastric tube is rinsed once with 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, or 20 mL of the liquid vehicle. In another example, the nasogastric tube is rinsed twice with 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, or 20 mL of the liquid vehicle.

In some embodiments, contemplated methods include a method of administering to the patient about 34 mg of pimavanserin once daily; wherein the administration comprises the steps of: a) opening a pharmaceutically acceptable capsule comprising pimavanserin; b) sprinkling the contents of the capsule onto about 5 mL to about 60 mL of a soft food vehicle at ambient or cold temperature; and c) orally delivering the contents of the capsule with the soft food vehicle to the patient.

In the methods provided herein, the ambient temperature may be United States Pharmacopeia (USP) controlled room temperature or from 20° C. to 25° C. The cold temperature can, for example, be USP controlled cold temperature or from 2° C. to 8° C.

In some embodiments of a method described herein, pimavanserin is stable in the liquid vehicle for at least 2 hours. For example, pimavanserin is stable in the liquid vehicle for at least 24 hours.

In other embodiments of a method described herein, pimavanserin is stable in the nasogastric tube for at least 2 hours. For example, pimavanserin is stable in the nasogastric tube for at least 24 hours.

The stability of pimavanserin in the liquid vehicle can be assessed by the Stability Assay as described in Example 1 of the present disclosure. In certain embodiments, the solution or mixture shows >95% pimavanserin at 2 hours compared to time 0. In certain embodiments, the assay of the solution or mixture shows >95% pimavanserin at 24 hours compared to time 0.

For example, the stability of pimavanserin in the nasogastric tube may be determined by the Nasogastric Tube Compatibility Study as described in Example 2 of the present disclosure. In some embodiments, the assay results of the solution or mixture are within 5% of the assay results of a control sample. In some embodiments, pimavanserin is stable in the nasogastric tube for at least 2 hours; or in another embodiment for at least 24 hours.

Contemplated herein is a method of treating a disorder in a patient in need thereof, the method comprising administering to the patient about 34 mg of pimavanserin via a feeding tube (e.g., an oral or enteral feeding tube) once daily, wherein pimavanserin is dissolved in or mixed with about 5 mL to about 60 mL of a liquid vehicle before the administration.

In another aspect, the present disclosure provides a method of treating hallucinations and delusions associated with Parkinson's disease in a patient in need thereof, the method comprising administering to the patient a pimavanserin composition via an oral or enteral feeding tube once daily, wherein the pimavanserin composition comprises about 34 mg pimavanserin and about 5 mL to about 60 mL of a liquid vehicle.

The contents of the capsule comprising 34 mg of pimavanserin with a liquid or soft food vehicle can be delivered to patients without clogging the feeding tube. The contents of the capsule comprising 34 mg of pimavanserin with a liquid or soft food vehicle is compatible with the feeding tube.

In certain embodiments of a method disclosed herein, the feeding tube is rinsed with an at least 5 mL of the liquid vehicle. In some embodiments, the feeding tube is rinsed with an additional 5 mL to 20 mL of a liquid vehicle. For example, the feeding tube is rinsed with 5 mL to 15 mL, or 5 mL to 10 mL. In other examples, the feeding tube is rinsed with 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, or 20 mL of the liquid vehicle.

In some embodiments of a method described herein, the liquid vehicle is selected from the group consisting of water, apple juice, applesauce, butter milk, coconut milk, cranberry juice, grapefruit juice, honey, maple syrup, milk, orange juice, pineapple juice, soybean milk, and vanilla shake, at ambient or cold temperature before the administration. In certain embodiments, the liquid vehicle selected from the group consisting of water, apple juice, vanilla shake, and orange juice. For example, the liquid vehicle is water. As another example, the liquid vehicle is applesauce or vanilla shake.

Contemplated soft food vehicles may be selected from the group consisting of apples (puree), applesauce, bananas (puree), orange juice, carrots (puree), chocolate pudding, fruit jellies, fruit jam, orange marmalade, a nut or seed butter such as peanut butter, sunflower seed butter or almond butter, rice pudding, strawberries (puree), strawberry jam, vanilla shake (e.g., vanilla Ensure®, and yogurt (e.g., plain (full-fat) yogurt). In some embodiments, the soft food vehicle may be selected from the group consisting of chocolate pudding, a nut or seed butter (such as peanut butter, sunflower seed butter or almond butter), and yogurt. In certain embodiments, the soft food vehicle is selected from the group consisting of applesauce, orange juice, and vanilla shake. 103. In other embodiments, the soft food vehicle is selected from the group consisting of apples (puree), applesauce, bananas (puree), carrots (puree), chocolate pudding, fruit jellies, fruit jam, orange marmalade, a nut or seed butter (such as peanut butter, sunflower seed butter or almond butter), rice pudding, strawberries (puree), strawberry jam, vanilla shake, and yogurt. In some embodiments, the soft food vehicle may be selected from the group consisting of applesauce, chocolate pudding, vanilla shake, and yogurt. Mixing drug product with liquids or soft foods may allow masking of an unpleasant taste, after-taste, smell and/or texture, and/or may facilitate swallowing the drug product. In certain embodiments, the soft foods described herein may not require chewing.

According to another embodiment of this disclosure, the contents of a capsule comprising 34 mg of pimavanserin with the liquid or soft food vehicle is stable when administered in via a nasogastric tube after holding for at least 5 minutes, or at least 10 minutes or at least 2 h.

The disclosure provides, in part, a method of treating a disorder in a patient in need of pimavanserin, wherein the patient is being treated concurrently with a strong CYP3A4 inhibitor, the method comprising administering to the patient about 10 mg of pimavanserin via a nasogastric tube once daily, wherein pimavanserin is dissolved in or mixed with about 5 mL to about 150 mL (e.g., about 5 mL to about 60 mL, about 5 mL to about 15 mL) of a liquid vehicle at ambient or cold temperature before the administration.

In another aspect, provided herein is a method of treating a disorder in a patient in need of pimavanserin, wherein the patient is being treated concurrently with a strong CYP3A4 inhibitor, the method comprising administering to the patient about 10 mg of pimavanserin via a nasogastric tube once daily, wherein pimavanserin is dissolved in or mixed with a liquid vehicle at ambient or cold temperature before the administration, wherein the solution or mixture is stable in the nasogastric tube at ambient or cold temperature.

Provided herein, in part, is a method of treating a disorder in a patient in need of pimavanserin, wherein the patient is being treated concurrently with a strong CYP3A4 inhibitor, the method comprising administering to the patient about 10 mg of pimavanserin once daily, wherein the administration comprises the steps of:
a) opening a pharmaceutically acceptable capsule comprising pimavanserin;
b) mixing the contents of the capsule with about 5 mL to about 150 mL (e.g., about 5 mL to about 60 mL, about 5 mL to about 15 mL) of a liquid vehicle or sprinkling the contents of the capsule onto about 5 mL to about 150 mL (e.g., about 5 mL to about 60 mL, about 5 mL to about 15 mL) of a soft food vehicle at ambient or cold temperature; and
c) delivering via a nasogastric tube the contents of the capsule with the liquid vehicle to the patient, or orally delivering the contents of the capsule with the soft food vehicle to the patient.

Also provided herein is a method of treating a disorder in a patient in need thereof, wherein the patient is being treated concurrently with a strong CYP3A4 inhibitor, the method comprising administering to the patient about 10 mg of pimavanserin via a feeding tube once daily, wherein pimavanserin is dissolved in or mixed with about 5 mL to about 150 mL (e.g., about 5 mL to about 60 mL, about 5 mL to about 15 mL) of a liquid vehicle before the administration. In some embodiments, the feeding tube is an oral feeding tube. In certain embodiments, the feeding tube is an enteral feeding tube.

In another aspect, the disclosure provides a method of treating hallucinations and delusions associated with Parkinson's disease in a patient in need thereof, wherein the patient is being treated concurrently with a strong CYP3A4 inhibitor, comprising administering to the patient a pimavanserin composition via a nasogastric tube once daily, wherein the pimavanserin composition comprises about 10 mg pimavanserin and about 5 mL to about 60 mL of a liquid vehicle.

Provided in the present disclosure includes a method of treating hallucinations and delusions associated with Parkinson's disease in a patient in need thereof, wherein the patient is being treated concurrently with a strong CYP3A4 inhibitor, the method comprising administering to the patient a pimavanserin composition via a nasogastric tube once daily, wherein the pimavanserin composition comprises about 10 mg pimavanserin and a liquid vehicle, wherein the composition is stable in the nasogastric tube at ambient or cold temperature.

Also provided herein is a method of treating hallucinations and delusions associated with Parkinson's disease in a patient in need thereof, wherein the patient is being treated concurrently with a strong CYP3A4 inhibitor, comprising administering to the patient about 10 mg pimavanserin once daily, wherein the administration comprises the steps of:
a) opening a pharmaceutically acceptable capsule comprising pimavanserin;
b) mixing the contents of the capsule with about 5 mL to about 60 mL of a liquid vehicle or sprinkling the contents of the capsule onto about 5 mL to about 60 mL of a soft food vehicle at ambient or cold temperature; and
c) delivering via a nasogastric tube the contents of the capsule with the liquid vehicle to the patient, or orally delivering the contents of the capsule with the soft food vehicle to the patient.

The present disclosure provides, in part, a method of treating hallucinations and delusions associated with Parkinson's disease in a patient in need thereof, wherein the patient is being treated concurrently with a strong CYP3A4 inhibitor, the method comprising administering to the patient a pimavanserin composition via an oral or enteral feeding tube once daily, wherein the pimavanserin composition comprises about 10 mg pimavanserin and about 5 mL to about 60 mL of a liquid vehicle.

A contemplated patient may have swallowing difficulties.

A contemplated patient that may be treated by disclosed methods is a patient that has psychosis secondary to a neurodegenerative disorder, for example Parkinson's disease psychosis or Alzheimer's disease psychosis, and is also suffering from dysphagia or swallowing difficulties.

Contemplated patients that may be treated by disclosed methods may also be patients concurrently suffering from hallucinations and delusions associated with Parkinson's disease and dysphagia or swallowing difficulties.

Unless otherwise indicated, a subject can be a mammal, a primate, a monkey or a human. In certain embodiments of the methods provided herein, the subject is a human subject.

In some embodiments, the human subject is a female human subject. In some embodiments, the human subject is a male human subject. In some embodiments, the human subject is a female or male human subject.

In some embodiments, the age of the human subject is 45 years or older, 50 years or older, or 55 years or old, or 60 years or older, or 65 years or older, or 70 years or older at baseline. In another embodiment, the age of the patient is 50 years or older. In another embodiment, the age of the patient is less than 50 years old.

### Compound

N-(4-Fluorophenylmethyl)-N-(1-methylpiperidin-4-yl)-N′-(4-(2-methylpropyloxy)phenylmethyl)carbamide is also known as pimavanserin; N-[(4-fluorophenyl)methyl]-N-(1-methyl-4-piperidinyl)-N′-[[4-(2-methylpropoxy)phenyl]methyl]-urea, 1-(4-fluorobenzyl)-1-(1-methylpiperidin-4-yl)-3-[4-(2-methylpropoxy)benzyl]urea or ACP-103 and is represented by the chemical formula:

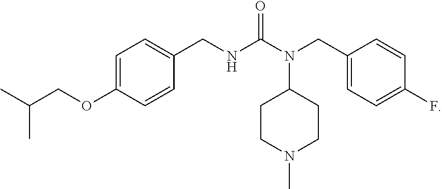 [SMILES: CC(C)COc1ccc(CNC(=O)N(Cc2ccc(F)cc2)C2CCN(C)CC2)cc1]

N-(4-Fluorophenylmethyl)-N-(1-methylpiperidin-4-yl)-N′-(4-(2-methylpropyloxy)phenylmethyl)carbamide maybe administered as a tartrate salt, which is urea, N-[(4-fluorophenyl)methyl]-N-(1-methyl-4-piperidinyl)-N′-[[4-(2-methylpropoxy)phenyl]methyl]-,(2R,3R)-2,3-dihydroxybutanedioate (2:1), and represented by the chemical formula:

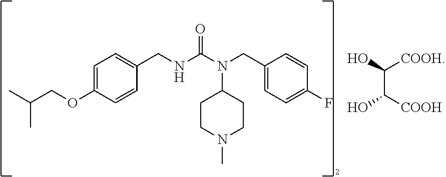

Pimavanserin (i.e., N-(4-Fluorophenylmethyl)-N-(1-methylpiperidin-4-yl)-N′-(4-(2-methylpropyloxy)phenylmethyl)carbamide) can be synthesized according to the method disclosed in Scheme I.

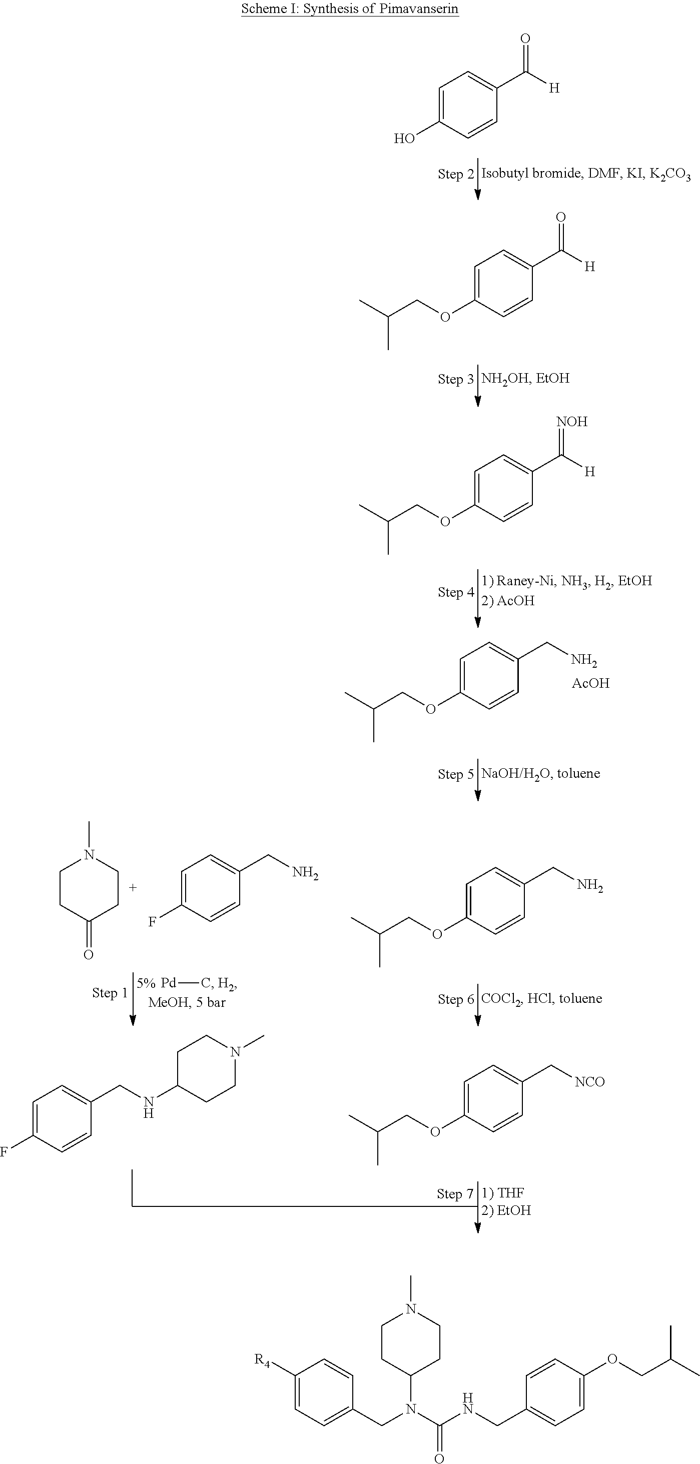

Pimavanserin can be obtained in a number of salt and crystalline forms. Exemplary pharmaceutically acceptable salts include the tartrate, hemi-tartrate, citrate, fumarate, maleate, malate, phosphate, succinate, sulphate, and edisylate (ethanedisulfonate) salts. Pimavanserin, and salts thereof including the aforementioned ions, among others, are described, for example, in International Patent Publication WO 2008/144326, U.S. Patent Publication Nos. 2006-0111399 and 2010-0305329, and International Patent Application WO2017015272, the entirety of which is incorporated herein by reference. In an embodiment provided herein, pimavanserin is the tartrate salt of pimavanserin. Several crystalline forms of the tartrate salt of pimavanserin are known. In an embodiment provided herein, pimavanserin is the crystalline form of the tartrate salt of pimavanserin Form A. In another embodiment, pimavanserin is the crystalline form of the tartrate salt of pimavanserin Form C.

In certain embodiments of the methods provided herein, pimavanserin or a pharmaceutically acceptable salt thereof is administered to the subject in an oral dosage form, such as, for example, a tablet, a capsule, or a lozenge and the like.

In some embodiments, the drug product is formulated with standard pharmaceutical excipients at a 34 mg pimavanserin (40 mg of pimavanserin tartrate) in capsules for oral administration. In certain embodiments the capsule formulation includes inactive ingredients magnesium stearate and microcrystalline cellulose. The following inactive ingredients can, for example, be present as components of the capsule shell: black iron oxide, FD&C blue #1, hypromellose, titanium dioxide, and yellow iron oxide.

In some embodiments, about 2 mg to about 80 mg of pimavanserin is a unit dose comprising pimavanserin tartrate. In other embodiments, about 4 mg to about 45 mg of pimavanserin is a unit dose comprising pimavanserin tartrate. In some embodiments, 34 mg of pimavanserin is a unit dose comprising pimavanserin tartrate. In certain embodiments, about 17 mg of pimavanserin is a unit dose comprising pimavanserin tartrate. In other embodiments, about 10 mg of pimavanserin is a unit dose comprising pimavanserin tartrate.

In certain embodiments of the methods provided herein, pimavanserin or a pharmaceutically acceptable salt thereof is orally administered to the subject in a capsule, wherein the amount of pimavanserin or pharmaceutically acceptable salt in the capsule is between 2 mg to 80 mg, between 5 mg to 45 mg, or between 9 mg to 42 mg.

In certain embodiments of the methods provided herein, pimavanserin or a pharmaceutically acceptable salt thereof is orally administered to the subject in a tablet, wherein the amount of pimavanserin or pharmaceutically acceptable salt in the tablet is between 2 mg to 80 mg, between 5 mg to 45 mg, or between 9 mg to 42 mg.

Disclosed method include administration of N-(4-fluorophenylmethyl)-N-(1-methylpiperidin-4-yl)-N′-(4-(2-methylpropyloxy)phenylmethyl)carbamide orally by table or capsule (34 mg of compounds). Tablets may contain 20 mg of pimavanserin tartrate, which is equivalent to 17 mg of pimavanserin free base, or 11.8 mg of pimavanserin tartrate, which is equivalent to 10 mg of pimavanserin free base. In the above formulations, pimavanserin is in crystalline and/or amorphous form.

Pimavanserin (i.e., N-(4-fluorophenylmethyl)-N-(1-methylpiperidin-4-yl)-N′-(4-(2-methylpropyloxy)phenylmethyl)carbamide) or a pharmaceutically acceptable salt thereof (e.g., tartrate salt) may be administered in the form of a capsule, e.g. a capsule may contain 40 mg of pimavanserin tartrate, which is equivalent to 34 mg of pimavanserin free base, or 20 mg of pimavanserin tartrate, which is equivalent to 17 mg of pimavanserin free base, or 11.8 mg of pimavanserin tartrate, which is equivalent to 10 mg of pimavanserin free base.

In some embodiments, the drug product is formulated with standard pharmaceutical excipients at a 17 mg strength (20 mg of pimavanserin tartrate) in immediate-release tablets for once-daily oral administration.

In some embodiments, the drug product is formulated with standard pharmaceutical excipients at a 10 mg strength (11.8 mg of pimavanserin tartrate) in immediate-release tablets for once-daily or twice-daily oral administration.

Pimavanserin once daily a 10 mg tablet can, for instance, be administered to the subject when a strong CYP3A4 inhibitor (e.g., ketoconazole) is also being administered to the subject.

In some embodiments, the dose and pharmaceutical composition of pimavanserin are those disclosed in International Application No. PCT/US2018/048096, which is incorporated herein for all purposes. Pimavanserin can be in a crystalline form. Such forms have been described as Forms A, B, C, D, etc., e.g. in WO 2006/037043, WO 2007/133802 and WO 2008/144326.

In some embodiments, the pharmaceutical composition comprises granulated pimavanserin tartrate, Form C which may include a small percentage of Form A, including a pharmaceutically acceptable diluent, binder or excipient, or combination thereof.

In some embodiments, the pharmaceutical compositions are provided as a two-piece hard shell capsules made of gelatin (fish, mammalian, or vegetable sourced) or other combinations. The two-piece hard shell capsules may contain the pimavanserin granules with a filler/diluent and/or lubricants. In some embodiments, the capsules are size 3 or 4 capsules. In some embodiments, the capsules are size 4 capsules. In some embodiment, the capsules are two-piece capsules of gelatin or hydroxypropyl methylcellulose (HPMC). Some commercial examples are VCAPS®, VCAPS® PLUS, CONI-SNAP® capsules marketed by Capsugel.

In some embodiments, the doses referred to herein refers to pimavanserin free base. In some embodiments, the doses referred to herein refers to pimavanserin tartrate Form C (e.g., 40 mg of pimavanserin tartrate, equivalent to 34 mg pimavanserin free base). In some embodiments, the doses refer to pimavanserin tartrate Form C encapsulated in capsules of size 3 or 4, such as capsules of size 4.

Provided are also embodiments wherein pimavanserin (granulated), microcrystalline cellulose, for example Avicel PH302 or an equivalent microcrystalline cellulose, and/or magnesium stearate, for example vegetable grade are encapsulated in a capsule of size 4, for example a two-piece capsule.

One embodiment of the compositions described herein includes pimavanserin tartrate granulation without binder, dried, and thereafter blended with less than 60% w/w microcrystalline cellulose such as Avicel PH302 or equivalent microcrystalline cellulose, and about 1% w/w magnesium stearate.

In some embodiments the compositions described herein comprise granulated pimavanserin and microcrystalline cellulose is at least 20% w/w microcrystalline cellulose, such as 30% w/w microcrystalline cellulose, such as 40% w/w microcrystalline cellulose, such as 50% w/w microcrystalline cellulose, such as 50-89% w/w microcrystalline cellulose, such as 20-94% w/w, such as 50-94% w/w, such as 57-94% w/w, such as 57-89% w/w microcrystalline cellulose, such as 57-79% w/w microcrystalline cellulose, or 57-60% w/w microcrystalline cellulose, or 57-59.5% w/w microcrystalline cellulose, or 58.5-59.5% w/w microcrystalline cellulose, or 59% w/w microcrystalline cellulose.

In some embodiments the compositions described herein comprise granulated pimavanserin and microcrystalline cellulose and magnesium stearate, such as 0.1-3% w/w, such as 0.5-2% w/w magnesium stearate, or 0.5-1.5% w/w magnesium stearate, or 1% w/w magnesium stearate.

In some embodiments the compositions described herein comprise granulated pimavanserin (5, 10, 20 or 34 mg) and microcrystalline cellulose is at least 20% w/w microcrystalline cellulose, such as 30% w/w microcrystalline cellulose, such as 40% w/w microcrystalline cellulose, such as 50% w/w microcrystalline cellulose, such as 50-89% w/w microcrystalline cellulose, such as 20-94% w/w, such as 50-94% w/w, such as 57-94% w/w, such as 57-89% w/w microcrystalline cellulose, such as 57-79% w/w microcrystalline cellulose, or 57-60% w/w microcrystalline cellulose, or 57-59.5% w/w microcrystalline cellulose, or 58.5-59.5% w/w microcrystalline cellulose, or 59% w/w microcrystalline cellulose and magnesium stearate, such as 0.1-3% w/w, such as 0.5-2% w/w magnesium stearate, or 0.5-1.5% w/w magnesium stearate, or 1% w/w magnesium stearate.

The compositions disclosed herein comprise pimavanserin and additional compatible excipients, e.g. sugars, sucrose, mannitol, sorbitol, polysaccharides (e.g. from corn, wheat, rice, potato), as well as pregelatinized or partially pregelatinized starches (e.g. STARCH 1500®), cellulose preparations such as microcrystalline cellulose (MCC) (e.g. AVICEL® PH 302, AVICEL® PH 102, VIVAPUR® 302, VIVAPUR® 102, EMCOCEL® HD 90), silicified microcrystalline cellulose (e.g. PROSOLV® 50, PROSOLV® 90, PROSOLV® HD90), lactose cellulose blends (e.g. CELLATOSE® 80, CELLATOSE® 90, PROSOLV® EASYtab SP), hydroxypropylmethyl cellulose, hydroxymethyl cellulose, polyvinylpyrrolidone, lubricants such as magnesium stearate, sodium stearyl fumarate, colloidal silicon dioxide, and talc.

Provided are also embodiments wherein 34 mg pimavanserin (granulated) (equivalent to 40 mg pimavanserin tartrate), microcrystalline cellulose, such as microcrystalline cellulose having a particle size distribution (D90) of 180-340 μm, for example Avicel PH302 or an equivalent microcrystalline cellulose, and/or magnesium stearate, for example vegetable grade are encapsulated in a capsule of size 4, for example a two-piece capsule.

Provided are also embodiments wherein 10 or 20 mg pimavanserin (granulated), microcrystalline cellulose, for example Avicel PH302 or an equivalent microcrystalline cellulose, and/or magnesium stearate, for example vegetable grade are encapsulated in a capsule of size 4, for example a two-piece capsule.

Provided are also embodiments wherein 34 mg pimavanserin (granulated), 59 mg microcrystalline cellulose, for example Avicel PH302 or an equivalent microcrystalline cellulose, and/or 1 mg magnesium stearate, for example vegetable grade are encapsulated in a capsule of size 4, for example a two-piece capsule. No other excipients were added.

Also provided is a pharmaceutical composition, comprising a capsule of pimavanserin and one or more pharmaceutically acceptable excipient(s) as provided herein, wherein the composition is formulated such that at least 80% of pimavanserin is released in 30 minutes upon administration to a subject.

Also provided is a pharmaceutical composition, comprising a capsule of pimavanserin and one or more pharmaceutically acceptable excipient(s) as provided herein, wherein the composition is formulated such that at least 80% of the pimavanserin is released from the composition within 30 minutes upon in vitro dissolution testing according to USP<711> (apparatus 1 (basket apparatus)).

In some embodiments, a pharmaceutically acceptable salt of pimavanserin is administered to the patient. In some specific embodiments, a tartrate salt of pimavanserin is administered to the patient.

In some embodiments, the pharmaceutically acceptable salt of pimavanserin comprises an anion selected from the group consisting of phosphate, sulphate, nitrate, diphosphate, besylate, bicarbonate, carbonate, clavulanate, edisylate, isothionate, borate, halide including e.g., chloride and bromide, nitrate, acetate, succinate, lactate, lactobionate, laurate, mandelate, malate, citrate, fumarate, maleate, oleate, oxalate, ascorbate, nicotinate, benzoate, mesylate, salicylate, stearate, tannate, tosylate, valerate, methanesulfonate, ethanesulfonate, benzenesulfonate, p-toluensulfonate, 2-ethane disulfonate, and naphthalenesulfonate.

The exact route of administration, dose, or frequency of administration would be readily determined by those skilled in the art and can be dependent on the age, weight, general physical condition, or other clinical symptoms specific to the patient to be treated.

In some embodiments, the tartrate salt of pimavanserin is administered daily. In some embodiments, the tartrate salt of pimavanserin is administered once daily. In some embodiments, the tartrate salt of pimavanserin is formulated for oral administration as a unit dose.

In an embodiment, pimavanserin is administered orally. In some embodiments, pimavanserin is administered via a nasogastric tube. In other embodiments, pimavanserin is administered via a feeding tube (e.g., oral feeding tube, an enteral feeding tube).

In an embodiment, pimavanserin is administered in a daily dose from about 0.5 mg to about 90 mg, or from about 8 mg to about 42 mg, or about 10 mg to about 60 mg.

In an embodiment, pimavanserin is administered in a daily dose from about 0.5 mg to about 120 mg, or from about 8 mg to about 42 mg, or about 10 mg to about 60 mg.

In certain embodiments, the above ranges are for pimavanserin free base. In certain embodiments, the above ranges are for a pharmaceutically acceptable salt, e.g., a tartrate salt of pimavanserin or any of the salts listed above.

In another embodiment, pimavanserin tartrate is administered in a daily dose of about 40 mg. In some embodiments, the daily dose is 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 80 mg, 88 mg or 102 mg. In an embodiment the daily dose of pimavanserin tartrate is administered once, twice or three times per day, for example a 40 mg dose of pimavanserin tartrate is administered once a day, or 20 mg pimavanserin tartrate is administered twice a day.

In another embodiment, pimavanserin is administered in a daily dose of about 34 mg. In some embodiments, the daily dose is 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 68 mg, 80 mg, 88 mg or 102 mg. In an embodiment the daily dose of pimavanserin is administered once, twice or three times per day, for example a 34 mg dose of pimavanserin is administered once a day, or 17 mg pimavanserin is administered twice a day, or a 10 mg dose pimavanserin is administered twice a day.

Alternative methods and equipment to be used in connection with the herein disclosed methods, compositions, capsules, tablets, soft food/liquid formulations and disclosures may be found in “Use of Liquids and/or Soft Foods as Vehicles for Drug Administration: General Considerations for Selection and In Vitro Methods for Product Quality Assessments”, a U.S. Department of Health and Human Services, Food and Drug Administration, Center for Drug Evaluation and Research (CDER) issued guidance for industry, e.g. in the July 2018 version, Pharmaceutical Quality/CMC.

### EXAMPLES

### General Procedures:

Analytical HPLC Stability Study experiments (Example 1) were performed on Agilent 1200 Series, or equivalent; Waters XBridge C18 column, 5 μm 150×4.6 mm; mobile phase: Solvent A: 50 mM ammonium bicarbonate, pH 8.0; Solvent B: acetonitrile/methanol, 80:20 (v/v); Flow rate: 1 mL/min; Inj. Volume 20 μL; Autosampler Temp. 5° C.; column temperature: 40° C., detection UV at 210 nm; sample concentration (as free base) assay: 0.068 mg/mL, impurity: 0.68 mg/mL; standard concentration (as free base) 0.068 mg/mL; PLOQ Std. 0.05% of 0.68 mg/mL; SST solution, 1% of 0.68 mg/mL (impurities A, C, D, E, F, and G); diluent for standards 80/20 (v/v) ethanol/water; diluent for samples, ethanol; run time 40 min; gradient method: 0 min mobile phase A (90%) and mobile phase B (10%) to 30 min mobile phase A (0%), and mobile phase B (100%); then 30.1 min to 40 min mobile phase A (90%), mobile phase B (10%).

Analytical HPLC Compatibility Study experiments (Example 2) were performed on Agilent 1200 Series, or equivalent; Waters XBridge C18 column, 5 μm 150×4.6 mm; mobile phase: Solvent A: 50 mM ammonium bicarbonate, pH 8.0; Solvent B: acetonitrile/methanol, 80:20 (v/v); Flow rate: 1 mL/min; Inj. Volume 60 μL; Autosampler Temp.: 5° C.; column temperature: 40° C.; the pooled limit of quantitation was 0.05% of 0.68 mg/mL; detection UV at 210 nm; sample concentration (as free base) assay: 0.034 mg/mL, impurity: 0.34 mg/mL; standard concentration (as free base) 0.034 mg/mL; PLOQ Std. 0.05% of 0.34 mg/mL; SST solution, 1% of 0.34 mg/mL (Impurities A, C, D, E, F, and G); diluent for standards 0.1N HCl; diluent for samples 0.1N HCl; run time, 40 min; gradient method: 0 min mobile phase A (90%) and mobile phase B (10%) to 30 min mobile phase A (0%), and mobile phase B (100%); then 30.1 min to 40 min mobile phase A (90%), mobile phase B (10%).

### Example 1: Stability Study

### Sample Preparation Procedure & Results—Pimavanserin Stability Assay

The stability of pimavanserin for administration via a nasogastric (NG) tube was assessed by sprinkling the content of one pimavanserin 34 mg capsule in water, applesauce, vanilla Ensure, and non-pulp orange juice. Samples were tested at time 0, 2, 4, and 24 hours after storage at ambient conditions. Samples were prepared in triplicate and average (n=6) accuracy/recovery results from the assay method were used at time 0.

For each sample, one capsule was weighed, and the content of the capsule dropped into a 500 mL volumetric flask containing the amount of vehicle specified in Table 1. The empty capsule shell was weighed and discarded. The flasks were gently swirled to ensure the capsule contents were visually incorporated with the vehicle. The flasks were kept at ambient temperature for each time point 2, 4, and 24 hours. Time 0 samples were processed immediately.

TABLE 1
Sample preparation for Assay

| Capsule | | | | | Non-Pulp |
|---|---|---|---|---|---|
| strength | Number of | Applesauce | Ensure | Water | Orange Juice |
| (mg) | capsules | (g) | (mL) | (mL) | (mL) |
| 34 | 1 | 40 | 60 | 40 | 60 |

At the specified time point, a stir bar and approximately 450 mL of diluent were added to the flask. The samples were stirred vigorously for 2 hours, and then left on the bench overnight. The following day the flasks were sonicated for approximately 15-30 minutes. The stir bar was removed and rinsed carefully with diluent into the flask, and then the flask was diluted to volume with diluent and mixed well by inversion. An aliquot of each solution was centrifuged for 5 minutes at 10000 rpm. The supernatant was transferred to a HPLC vial and injected for analysis.

### Results

As shown in Table 2, the stability study demonstrated the ability to deliver 34 mg of pimavanserin with >95% recovery within 24 hours after the contents of one pimavanserin 34 mg capsule was dispersed in each of the vehicles: water applesauce, vanilla Ensure, or orange juice. The stability of pimavanserin in all vehicles was acceptable over 24 hours at ambient room temperature, Assay values up to 24 hours for individual capsules were within 5% of time 0, and no significant change in impurity profile was observed in any vehicle. An assay value of 96.3% was recorded in one replicate preparation (applesauce) after 24 hours of storage, which differed by 5.6% from time 0 and was above the 5% acceptance criteria specified by the protocol. This value was accepted based on the expected recovery range of 94.4% to 104.4% obtained for individual capsules (n=24) during the method accuracy/precision experiments.

TABLE 2
In-use Stability of Pimavanserin from 34 mg Capsules

| | | | Absolute |
|---|---|---|---|
| | | | Difference |
| | | Time 0 | from Time 0 |
| Sample/Time | Assay | (%) | (%) |
| Water Assay T = 2 h | 97.7 | 98.9 | 1.2 |
| Water Assay T = 4 h | 95.6 | | 3.3 |
| Water Assay T = 24 h | 99.3 | | 0.4 |
| Applesauce Assay T = 2 h | 99.9 | 101.9 | 2.0 |
| Applesauce Assay T = 4 h | 97.7 | | 4.2 |
| Applesauce Assay T = 24 h | 96.3 | | 5.6 |
| Orange juice Assay T = 2 h | 99.2 | 100.3 | 1.1 |
| Orange juice Assay T = 4 h | 98.0 | | 2.3 |
| Orange juice Assay T = 24 h | 98.3 | | 2.0 |
| Ensure Assay T = 2 h | 100.3 | 100.0 | 0.3 |
| Ensure Assay T = 4 h | 100.7 | | 0.7 |
| Ensure Assay T = 24 h | 98.1 | | 1.9 |

### Sample Preparation Procedure & Results—Impurity Analysis

Samples for the assessment of the degradation products stability from solutions or mixtures of pimavanserin in vehicles were prepared as follows. Four samples (n=4) were prepared for each vehicle as follows.

For each sample, one capsule was weighed, and the content of each capsule was dropped into a 50 mL volumetric flask and the empty capsule shell was weighed and discarded. The specified amount of each vehicle was added as specified in Table 3, below.

TABLE 3
Sample preparation for Impurity Assay

| Capsule | | | | | Non-Pulp |
|---|---|---|---|---|---|
| strength | Number of | Applesauce | Ensure | Water | Orange Juice |
| (mg) | capsules | (g) | (mL) | (mL) | (mL) |
| 34 | 1 | 5 | 5 | 5 | 5 |

The flasks were gently swirled to ensure the capsule contents were visually incorporated with the vehicle. The flasks were kept on the bench at ambient temperature for each time point: 2, 4, and 24 hours. Time 0 samples were processed immediately. At the specified time point a stir bar and approximately 45 mL of diluent were added to the flask. The solution was stirred vigorously for 2 hours, and the flask was left on the bench overnight. The next day the flasks were sonicated for approximately 30 minutes. The stir bar was removed and rinsed carefully with diluent into the flask, and the flask was diluted to volume with diluent and mixed well by inversion. An aliquot of each solution was centrifuged for 5 minutes at 10000 rpm. The supernatant was transferred to a HPLC vial, and injected for analysis.

A second dilution from the impurity stock was used for assay testing (in order to demonstrate mass balance) as shown in Table 4 below. Samples were prepared by transferring the stock volume using a volumetric pipet into a 20 mL volumetric flask, diluted to volume with diluent, and mixed well by inversion. An aliquot of each solution was centrifuged for 5 minutes at 10000 rpm. The supernatant was transferred to a HPLC vial and injected for analysis.

TABLE 4
Dilution scheme

| | Stock | Stock | Total | Final |
|---|---|---|---|---|
| Solution | Volume | Concentration | Volume | Concentration |
| No. | (mL) | (mg/mL) | (mL) | (mg/mL) |
| Impurity | N/A | N/A | 50 mL | 0.68 |
| Assay | 2.0 | 0.68 | 20 | 0.068 |

### Results

As shown in Table 5. the stability results for pimavanserin impurities recovered from various food vehicles for individual and total impurities were consistent between all timepoints for each vehicle. Two additional unknown impurities <0.1% were observed in the samples extracted from vanilla Ensure at time 0 and did not change significantly during stability storage.

TABLE 5
In-use Stability for Pimavanserin Impurities

| | | Relative | | | | | |
|---|---|---|---|---|---|---|---|
| | Run Time | Retention | | T = 0% | T = 2 h | T = 4 h | T = 24 h |
| Sample | (min) | Time | Impurity | w/w | % w/w | % w/w | % w/w |
| Water imp. | 8.98 | 0.44 | D | 0.23 | 0.24 | 0.23 | 0.25 |
| | 20.06 | 0.99 | Unknown | 0.10 | 0.10 | 0.10 | 0.10 |
| | | | RRT | | | | |
| | | | 0.99 | | | | |
| | | | Total Imp. | 0.33 | 0.34 | 0.33 | 0.35 |
| Applesauce | 8.91 | 0.44 | D | 0.19 | 0.21 | 0.22 | 0.22 |
| Imp. | 19.95 | 0.99 | Unknown | 0.09 | 0.10 | 0.10 | 0.09 |
| | | | RRT | | | | |
| | | | 0.99 | | | | |
| | | | Total Imp. | 0.28 | 0.31 | 0.32 | 0.31 |
| Orange Juice | 8.92 | 0.44 | D | 0.17 | 0.17 | 0.17 | 0.17 |
| Imp. | 20.07 | 0.99 | Unknown | 0.09 | 0.09 | 0.09 | 0.09 |
| | | | RRT | | | | |
| | | | 0.99 | | | | |
| | | | Total Imp. | 0.26 | 0.26 | 0.26 | 0.26 |
| Ensure Imp. | 8.95 | 0.44 | D | 0.22 | 0.20 | 0.23 | 0.23 |
| | 18.31 | 0.90 | Unknown | 0.08 | 0.08 | 0.08 | 0.07 |
| | | | RRT | | | | |
| | | | 0.90 | | | | |
| | 20.06 | 0.99 | Unknown | 0.09 | 0.10 | 0.09 | 0.10 |
| | | | RRT | | | | |
| | | | 0.99 | | | | |
| | 22.29 | 1.10 | Unknown | 0.06 | 0.05 | 0.06 | 0.06 |
| | | | RRT | | | | |
| | | | 1.10 | | | | |
| | | | Total Imp. | 0.45 | 0.43 | 0.46 | 0.46 |

Assay results obtained from diluted impurity samples are shown in Table 6. All assay results were within 5% from time 0 and demonstrated ability to deliver the expected dose (>95% recovery) within 24 hours at ambient room temperature after being dispersed in a tablespoon of water, applesauce, vanilla Ensure, or orange juice.

TABLE 6
In-Use Stability for Pimavanserin Impurities -
Assay Results from Diluted Impurity Samples

| | | | Absolute |
|---|---|---|---|
| | | | Difference |
| | Assay | Time 0 | from Time 0 |
| Sample/Time | (%) | (%) | (%) |
| Water Assay from Imp. T = 0 | 95.6 | 98.9 | 3.3 |
| Water Assay from Imp. T = 2 h | 98.2 | | 0.7 |
| Water Assay from Imp. T = 4 h | 97.1 | | 1.8 |
| Water Assay Imp. T = 24 h | 97.5 | | 1.4 |
| Applesauce Assay from Imp. T = 0 | 97.5 | 101.9 | 4.4 |
| Applesauce Assay from Imp. T = 2 h | 99.4 | | 2.5 |
| Applesauce Assay from Imp. T = 4 h | 104.1 | | 2.2 |
| Applesauce Assay from Imp. T = 24 h | 99.5 | | 2.4 |
| Orange juice Assay from Imp. T = 0 | 97.7 | 100.3 | 2.6 |
| Orange juice Assay from Imp. T = 2 h | 97.5 | | 2.8 |
| Orange juice Assay from Imp. T = 4 h | 96.4 | | 3.9 |
| Orange juice Assay from Imp. T = 24 h | 99.2 | | 1.1 |
| Ensure Assay from Imp. T = 0 | 97.1 | 100.00 | 2.9 |
| Ensure Assay from Imp. T = 2 h | 94.6 | | 5.4 |
| Ensure Assay from Imp. T = 4 h | 97.9 | | 2.1 |
| Ensure Assay from Imp. T = 24 h | 97.3 | | 2.7 |

### Example 2: Nasogastric Tube Compatibility Study

Three samples of pimavanserin in water (n=3) were prepared as follows. The content of one capsule was dropped into a 100 mL bottle, and the empty capsule shell was discarded. Approximately 40 mL of water was added into the bottle, and the bottle was swirled until the contents were disintegrated. Syringes were secured approximately 1 meter above the benchtop, and the plungers were removed. The Hydromer coating on the weighted top of the NG tube was activated by submerging the weight assembly in water for 10 seconds. The NG tube was flushed with 20 mL of water, and remaining water in the syringe and the NG tube was expelled with the help of the syringe plunger. The weighted tip of the NG tube was placed into 100 mL volumetric flasks on the benchtop.

For each sample, the aqueous dispersion was slowly poured into the syringe, allowing it to drain through the NG tube under gravity into the 100 mL collection volumetric flask. The original bottle containing the aqueous suspension of pimavanserin capsules was rinsed twice with 15 mL of water into the same syringe/NG tube system. The dispersion remaining in the syringe and NG tube was expelled with the help of the syringe plunger. Each flask was diluted to volume with 0.33N HCl (to make a final 0.1 N HCl concentration in the flask). The samples were sonicated for 15 minutes with intermediate shaking. A stir bar was added to the flask and the solution was stirred for 2 hours. An aliquot of each solution was centrifuged for 5 minutes at 10000 rom. The supernatant was transferred to a HPLC vial and injected for impurity analysis.

A second dilution from impurity stock was used for assay testing as shown in Table 7 below. Samples were prepared by transferring the stock solution using a 2.0 mL volumetric pipet into a 20 mL volumetric flask, diluted to volume with 0.1 N HCl, and mixed well by inversion. An aliquot of each solution was centrifuged for 5 minutes at 10000 rpm. The supernatant was transferred to a HPLC vial and injected for analysis.

TABLE 7
Dilution scheme

| | Stock | Stock | Total | Final |
|---|---|---|---|---|
| Solution | Volume | Concentration | Volume | Concentration |
| No. | (mL) | (mg/mL) | (mL) | (mg/mL) |
| Impurity | N/A | N/A | 100 mL | 0.34 |
| Assay | 2.0 | 0.34 | 20 | 0.034 |

A “control” sample was prepared following the sample preparation procedure described above except it was not passed through a syringe/NG system. The content of one capsule was dropped into a 100 mL volumetric flask, and the empty capsule shell was discarded. Next 70 mL of water was added into the flask and swirled until the contents were completely disintegrated. The flask was diluted to volume with 0.33 N HCl (to make final 0.1N HCl concentration). The sample was sonicated for 15 minutes with intermediate shaking. A stir bar was dropped into the flask, and the solution was stirred for 2 hours. An aliquot was taken and centrifuged for 5 minutes at 10000 rpm. The supernatant was transferred to a HPLC vial and injected for impurity analysis.

A water only “Rinse Control” sample was prepared following the sample preparation procedure described above, except only water was passed through syringe/NG tube system. The syringe was secured approximately 1 meter above the benchtop, and the plungers were removed. The Hydromer coating on the weighted tips of the NG tube was activated by submerging the weight assembly in water for 10 seconds. The NG tube was flushed with 20 mL of water by collecting the 11^{th} and 20^{th} mL of the rinse for analysis. Remaining water in the syringe and the NG tube was expelled using the syringe plunger. The weighted tip of the NG tube was placed into 100 mL volumetric flasks on the benchtop. Approximately 70 mL of water was slowly poured into the syringe with the plunger removed, allowing it to drain through the NG tube under gravity into the 100 mL collection volumetric flask. Water remained in the syringe and the NG tube was expelled with the aid of the syringe plunger. The collection flask was diluted to volume with 0.33 N HCl and mixed well. An aliquot of the solution was centrifuged for 5 minutes at 10000 rpm. The supernatant was transferred to a high pressure liquid chromatography (HPLC) vial and injected for analysis.

### Results

Assay and impurities data obtained for 3 compatibility samples and the data obtained for “control” sample are shown below in Table 8 and Table 9. The compatibility study of the aqueous suspension of pimavanserin 34 mg capsules with a syringe/NG tube device demonstrated the ability to administer an aqueous dispersion of pimavanserin 34 mg through a NG tube with acceptable recovery. The assay results between the control sample and mean results from 3 compatibility samples exposed to the NG tube matched within 2%. In addition, the impurity profile of compatibility samples matched that obtained for a control sample.

TABLE 8
Assay results from NG Tube Compatibility Study

| | | | % Relative |
|---|---|---|---|
| | Assay | Mean % | Standard Deviation |
| Sample | (%) | (n = 6) | (n = 3) |
| NG Tube N = 1 | 92.2 | 93.8 | 2.4 |
| NG Tube N = 2 | 92.7 | | |
| NG Tube N = 3 | 96.3 | | |
| NG Tube Control | 91.5 | — | — |

TABLE 9
Impurity results ≥ 0.05% w/w

| | | | | | | | NG |
|---|---|---|---|---|---|---|---|
| Retention | Relative | | NG | NG | NG | NG | Tube |
| time | time | | Tube | Tube | Tube | Tube | Mean |
| (min) | retention | Name | Control | N = 1 | N = 2 | N = 3 | (n = 3) |
| 7.30 | 0.36 | RRT-0.36 | 0.05 | 0.05 | 0.06 | — | 0.06 |
| 8.82 | 0.43 | Imp-D | 0.13 | 0.13 | 0.14 | 0.13 | 0.13 |
| 10.75 | 0.53 | RRT-0.53 | — | — | — | — | — |
| 10.95 | 0.54 | RRT-0.54 | 0.07 | 0.07 | 0.07 | — | 0.07 |
| 11.03 | 0.54 | Imp-A | — | — | — | — | — |
| 12.30 | 0.60 | Imp-F | — | — | — | — | — |
| 14.29 | 0.70 | RRT-0.70 | — | — | — | — | — |
| 17.04 | 0.84 | Imp-G | — | — | — | — | — |
| 17.28 | 0.85 | RRT-0.85 | — | 0.05 | — | — | 0.05 |
| 18.42 | 0.90 | RRT-0.90 | — | — | — | — | — |
| 19.60 | 0.96 | RRT-0.96 | 0.06 | 0.05 | 0.06 | — | 0.06 |
| 20.09 | 0.99 | Imp-C | — | — | — | — | — |
| 20.17 | 0.99 | RRT-0.99 | 0.07 | 0.07 | 0.07 | 0.05 | 0.06 |
| 22.36 | 1.10 | RRT-1.10 | — | — | — | — | — |
| 22.58 | 1.11 | RRT-1.11 | — | — | 0.05 | — | 0.05 |
| 23.92 | 1.17 | Imp-E | — | — | — | — | — |

| | | | | | |
|---|---|---|---|---|---|
| Total degradation products and | 0.38 | 0.42 | 0.45 | 0.18 | 0.48 |
| impurities | | | | | |

RRT = relative retention time

### Conclusions

The results reported here found no effect in in vitro administration through a NG tube on the stability of pimavanserin when the contents of the capsules were added to different vehicles. Recovery of pimavanserin was also not affected by NG tube administration, with >95% of the administered dose recovered. Very low levels of unknown impurities (<0.1%) were found in either the stability or recovery portions of the studies conducted.

### Example 3. An Open-Label Crossover Study to Assess the Palatability and Swallowability of Pimavanserin when Mixed with Food Vehicles in Healthy Elderly Adults

Studies are conducted to evaluate the palatability of pimavanserin when mixed with selected food vehicles in healthy elderly adults and the swallowability of pimavanserin when mixed with selected food vehicles in healthy elderly adults. The mouthfeel (texture), smell, aftertaste, and bitterness of pimavanserin when mixed with selected food vehicles in healthy elderly adults are also evaluated. Furthermore, the ease of manipulation of the pimavanserin 34 mg capsule for emptying its contents onto food vehicles is explored.

### Subjects

Up to 20 non-smoking, healthy elderly subjects >60 years of age as follows: Part 1: Two subjects (1 male and 1 female) in Part 1a, and optionally an additional 2 subjects (1 male and 1 female) in Part 1b.
Part 2: Sixteen subjects (8 males and 8 females).
Subjects with a known personal or family history of long QT syndrome or family history of sudden cardiac death are excluded.

### Methods

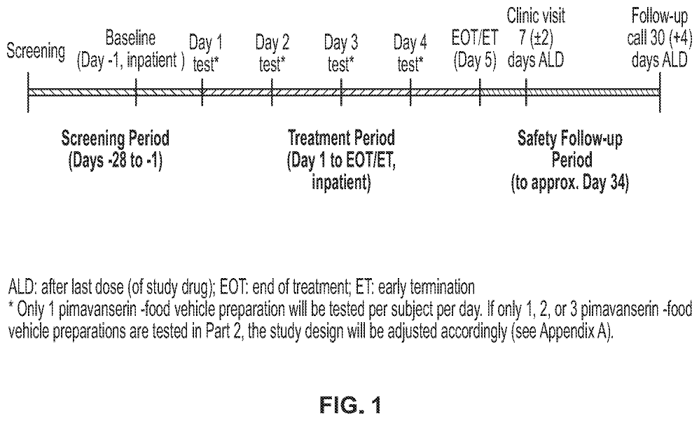

Pimavanserin 34 mg capsule contents are mixed into food vehicles. Up to four food vehicles are evaluated with each subject, one per day, presented in the morning. Each subject will receive up to four 34 mg doses of pimavanserin, one per day. A schematic of the study design is shown in FIG. 1.

The study will have 3 periods for each subject: Screening Period (2-28 days, includes Screening and Baseline days), Treatment Period (5 days, first treatment [Day 1] through end of treatment/early termination [EOT/ET; the day after the last dose of study drug, nominally Day 5]), and Safety Follow-up Period (30 [+4] days).

Eligible subjects will report to the clinic on Day-1 (Baseline) for baseline assessments and will fast overnight to begin the Treatment Period the next day.

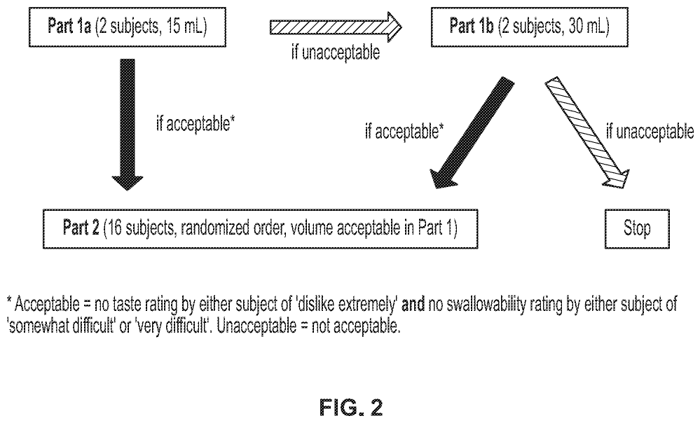

The Treatment Period will be conducted as described below and will vary depending on the part of the study (Part 1 vs. Part 2). As illustrated in FIG. 2, each of the 4 food vehicles will progress, or be stopped, based on their test results in the various parts of the study, independently of the test results obtained with the other food vehicles.

Part 1: Two (2) subjects will be used to assess the palatability and swallowability of each of 4 pimavanserin—food vehicle preparations using a small volume of food vehicle (e.g., 15 mL [1 tablespoon], Part 1a). All pimavanserin—food vehicle preparations not found acceptable based on either taste or swallowability ratings using the small volume will be tested using a larger volume of food vehicle (30 mL [2 tablespoons]), each with another 2 unique subjects (Part 1b).

Part 2: All pimavanserin—food vehicle preparations that are found to be acceptable in Part 1 (in either volume) will be tested in each of 16 additional subjects using the volume found acceptable in Part 1.

Note that based on the above, it is possible to have anywhere from 1 to 4 food vehicles progress to Part 2, and for some of food vehicles to be tested in Part 2 using the smaller volume (e.g., 15 mL) and some food vehicles to be tested in Part 2 using the larger volume (e.g., 30 mL). It is also possible that no food vehicle will be found acceptable in Part 1, in which case the study will not progress to Part 2.

On each of Days 1 to 4 (the treatment days), subjects will assess a single, different food vehicle combined with 34 mg pimavanserin using 5-point rating scales for taste, mouthfeel (texture), smell, aftertaste, bitterness, and swallowability. Aftertaste will also be assessed at EOT/ET (nominally Day 5). The testing of a pimavanserin—food vehicle preparation may be delayed by up to 2 days if aftertaste from a pimavanserin—food vehicle preparation tested earlier persists. The ratings are shown in Table 10.

The 4 treatments to be administered, with pimavanserin capsules opened and the contents mixed into a food vehicle are:

A: pimavanserin 34 mg+applesauce
B: pimavanserin 34 mg+chocolate pudding
C: pimavanserin 34 mg+plain (full-fat) yogurt
D: pimavanserin 34 mg+vanilla Ensure®

Assuming 4 pimavanserin—food vehicle preparations are tested in Part 2, this part of the study will use a four-way Latin square crossover design with 4 treatments, 4 periods and 4 sequences balanced for first-order carryover effects. Subjects will be randomly assigned to 1 of the 4 sequences in a 1:1:1:1 ratio using permuted blocks. The randomization (Day 1) will be stratified by sex. If only 1, 2, or 3 pimavanserin—food vehicle preparations are tested in Part 2, the study design will be adjusted accordingly. Subjects who discontinue prior to completion of pimavanserin—food vehicle preparation testing will not be replaced. In Part 1 of the study, the pimavanserin—food vehicle preparation sequences will be used as follows: first subject (Part 1a), sequence 1; second subject (Part 1a), sequence 2; third subject (Part 1b), sequence 3; fourth subject (Part 1b), sequence 4. The sequences are shown in Table 11.

In Part 2 of the study only, during the afternoon of each treatment day, ease of capsule manipulation assessment will be performed. This will assess the ability of the subject to open the capsule and empty its contents. Ease of capsule manipulation assessment will not include mixing with food vehicle or consumption of drug.

TABLE 10
Rating scales

| | | | | | | | Ease of |
|---|---|---|---|---|---|---|---|
| | | | | | | | capsule |
| Score | Taste | Mouthfeel | Smell | Aftertaste | Bitterness | Swallowability | manipulation |
| 1 | like | like | like | like | no | very easy | very easy |
| | extremely | extremely | extremely | extremely | bitterness | | |
| 2 | like | like | like | like | mild | somewhat | somewhat |
| | moderately | moderately | moderately | moderately | bitterness | easy | easy |
| 3 | neither | neither | neither | neither | moderate | neither easy | neither easy |
| | like nor | like nor | like nor | like nor | bitterness | nor | nor |
| | dislike | dislike | dislike | dislike | | difficult | difficult |
| 4 | dislike | dislike | dislike | dislike | severe | somewhat | somewhat |
| | moderately | moderately | moderately | moderately | bitterness | difficult | difficult |
| 5 | dislike | dislike | dislike | dislike | extreme | very | very |
| | extremely | extremely | extremely | extremely | bitterness | difficult | difficult |

TABLE 11
Sequences of pimavanserin

| Sequence | Day 1 | Day 2 | Day 3 | Day 4 |
|---|---|---|---|---|
| 1 | B | A | C | D |
| 2 | C | B | D | A |
| 3 | D | C | A | B |
| 4 | A | D | B | C |

### Endpoints

Primary endpoints of this study are rating of palatability on a 1- to 5-point scale for taste (like extremely, like moderately, neither like nor dislike, dislike moderately, dislike extremely) and Rating of swallowability on a 1- to 5-point scale (very easy, somewhat easy, neither easy nor difficult, somewhat difficult, very difficult). The ratings are described in Table 10.

Secondary endpoints of this study are ratings of mouthfeel (texture), smell, aftertaste, and bitterness on 1- to 5-point scales; rating of ease of capsule manipulation by subject on a 1- to 5-point scale (very easy, somewhat easy, neither easy nor difficult, somewhat difficult, very difficult); and ability of subject to open capsule and empty contents (binary output) as judged by site staff.

Although the invention has been described with reference to embodiments and examples, it should be understood that numerous and various modifications can be made without departing from the spirit of the invention. Accordingly, the invention is limited only by the following claims. All publications, patents, and patent applications cited in this specification are incorporated herein by reference as if each such publication, patent or patent application were specifically and individually indicated to be incorporated herein by reference.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A method of treating a disorder in a patient in need of pimavanserin, the method comprising administering to the patient about 2 mg to about 80 mg of pimavanserin once daily, wherein pimavanserin is dissolved in or mixed with about 5 mL to about 150 mL of a liquid vehicle at ambient or cold temperature before the administration.

2. The method of claim 1, wherein the disorder is a psychosis secondary to a neurodegenerative disorder.

3. The method of claim 2, wherein the disorder is a psychosis secondary to Parkinson's disease, Alzheimer's disease, vascular dementia, or Lewy body dementia.

4. The method of claim 1, wherein the disorder is schizophrenia or major depressive disorder.

5. The method of claim 1, wherein the disorder is a negative symptom of schizophrenia.

6. The method of any one of claims 1-5, wherein the method comprises administering to the patient about 5 mg to about 45 mg of pimavanserin.

7. The method of any one of claims 1-6, wherein the method comprises administering to the patient about 34 mg of pimavanserin.

8. The method of any one of claims 1-6, wherein the method comprises administering to the patient about 20 mg of pimavanserin.

9. The method of any one of claims 1-6, wherein the method comprises administering to the patient about 17 mg of pimavanserin.

10. The method of any one of claims 1-6, wherein the method comprises administering to the patient about 10 mg of pimavanserin, wherein the patient is being treated concurrently with a strong CYP3A4 inhibitor.

11. The method of any one of claims 1-10, wherein pimavanserin is administered to the patient via an enteral feeding tube.

12. The method of claim 11, wherein the enteral feeding tube is a nasogastric tube.

13. The method of claim 11, wherein the enteral feeding tube is an orogastric tube.

14. The method of claim 11, wherein the enteral feeding tube is a nasoenteric tube.

15. The method of claim 11, wherein the enteral feeding tube is an oroenteric tube.

16. The method of any one of claims 1-15, wherein pimavanserin is dissolved in or mixed with about 5 mL to about 100 mL of the liquid vehicle.

17. The method of any one of claims 1-16, wherein pimavanserin is dissolved in or mixed with about 5 mL to about 60 mL of the liquid vehicle.

18. The method of any one of claims 1-17, wherein pimavanserin is dissolved in or mixed with about 5 mL to about 15 mL of the liquid vehicle.

19. The method of any one of claims 1-18, wherein pimavanserin is dissolved in or mixed with about 15 mL of the liquid vehicle.

20. The method of any one of claims 1-19, wherein pimavanserin is dissolved in or mixed with about 30 mL of the liquid vehicle.

21. The method of any one of claims 1-20, wherein the ambient temperature is USP controlled room temperature or from 20° C. to 25° C.

22. The method of any one of claims 1-21, wherein the cold temperature is USP controlled cold temperature or from 2° C. to 8° C.

23. The method of any one of claims 1-22, wherein the nasogastric tube is rinsed with an additional 5 mL to 20 mL of the liquid vehicle.

24. The method of any one of claims 1-23, wherein pimavanserin is stable in the liquid vehicle for at least 24 hours.

25. The method of claim 24, wherein the stability of pimavanserin is determined by the Stability Assay.

26. The method of claim 25, wherein the assay of the solution or mixture shows >95% pimavanserin at 24 hours compared to time 0.

27. The method of claim any one of claims 1-26, wherein the patient has swallowing difficulties.

28. A method of treating a disorder in a patient in need of pimavanserin, the method comprising administering to the patient about 2 mg to about 80 mg of pimavanserin via an enteral feeding tube once daily, wherein pimavanserin is dissolved in or mixed with a liquid vehicle at ambient or cold temperature before the administration, wherein the solution or mixture is stable in the enteral feeding tube at ambient or cold temperature.

29. The method of claim 28, wherein the disorder is a psychosis secondary to a neurodegenerative disorder.

30. The method of claim 28, wherein the disorder is schizophrenia or major depressive disorder.

31. The method of claim 28, wherein the disorder is a negative symptom of schizophrenia.

32. The method of any one of claims 28-31, wherein the method comprises administering to the patient about 5 mg to about 45 mg of pimavanserin.

33. The method of any one of claims 28-32, wherein the method comprises administering to the patient about 34 mg of pimavanserin.

34. The method of any one of claims 28-32, wherein the method comprises administering to the patient about 20 mg of pimavanserin.

35. The method of any one of claims 28-32, wherein the method comprises administering to the patient about 17 mg of pimavanserin.

36. The method of any one of claims 28-32, wherein the method comprises administering to the patient about 10 mg of pimavanserin.

37. The method of any one of claims 28-36, wherein the enteral feeding tube is a nasogastric tube.

38. The method of any one of claims 28-36, wherein the enteral feeding tube is an orogastric tube.

39. The method of any one of claims 28-36, wherein the enteral feeding tube is a nasoenteric tube.

40. The method of any one of claims 28-36, wherein the enteral feeding tube is an oroenteric tube.

41. The method of any one of claims 28-40, wherein the ambient temperature is USP controlled room temperature or from 20° C. to 25° C.

42. The method of any one of claims 28-41, wherein the cold temperature is USP controlled cold temperature or from 2° C. to 8° C.

43. The method of any one of claims 28-42, wherein pimavanserin is stable in the enteral feeding tube for at least 2 hours.

44. The method of claim 43, wherein pimavanserin is stable in the enteral feeding tube for at least 24 hours.

45. The method of any one of claims 28-44, wherein the stability of pimavanserin is determined by the Nasogastric Tube Compatibility Study.

46. The method of claim 45, wherein the assay results of the solution or mixture are within 5% of the assay results of a control sample.

47. The method of any one of claims 28-46, wherein the patient has swallowing difficulties.

48. A method of treating a disorder in a patient in need of pimavanserin, the method comprising administering to the patient about 2 mg to about 80 mg of pimavanserin once daily, wherein the administration comprises the steps of:
a) opening a pharmaceutically acceptable capsule comprising pimavanserin;
b) mixing the contents of the capsule with about 5 mL to about 150 mL of a liquid vehicle or sprinkling the contents of the capsule onto about 5 mL to about 150 mL of a soft food vehicle at ambient or cold temperature; and
c) delivering to the patient, optionally via an enteral feeding tube, the contents of the capsule with the liquid vehicle to the patient, or orally delivering the contents of the capsule with the soft food vehicle to the patient.

49. A method of treating a disorder in a patient in need of pimavanserin, the method comprising administering to the patient about 2 mg to about 80 mg of pimavanserin once daily, wherein the administration comprises the steps of:
a) opening a pharmaceutically acceptable capsule comprising pimavanserin;
b) mixing the contents of the capsule with about 5 mL to about 150 mL of a liquid vehicle or sprinkling the contents of the capsule onto about 5 mL to about 150 mL of a soft food vehicle at ambient or cold temperature; and
c) orally delivering the contents of the capsule with the soft food vehicle to the patient.

50. A method of treating a disorder in a patient in need of pimavanserin, the method comprising administering to the patient about 2 mg to about 80 mg of pimavanserin once daily, wherein the administration comprises the steps of:
a) crushing a pharmaceutically acceptable tablet comprising pimavanserin;
b) mixing the crushed tablet with about 5 mL to about 150 mL of a liquid vehicle or sprinkling the crushed tablet onto about 5 mL to about 150 mL of a soft food vehicle at ambient or cold temperature; and
c) delivering to the patient, optionally via an enteral feeding tube, the crushed tablet with the liquid vehicle to the patient, or orally delivering the crushed tablet with the soft food vehicle to the patient.

51. A method of treating a disorder in a patient in need of pimavanserin, the method comprising administering to the patient about 2 mg to about 80 mg of pimavanserin once daily, wherein the administration comprises the steps of:
a) crushing a pharmaceutically acceptable tablet comprising pimavanserin;
b) mixing the crushed tablet with about 5 mL to about 150 mL of a liquid vehicle or sprinkling the crushed tablet onto about 5 mL to about 150 mL of a soft food vehicle at ambient or cold temperature; and
c) orally delivering the crushed tablet with the soft food vehicle to the patient.

52. The method of any one of claims 48-51, wherein the contents of the capsule or the crushed tablet with the soft vehicle is orally delivered to the patient via an enteral feeding tube.

53. The method of any one of claims 48-52, wherein the disorder is a psychosis secondary to a neurodegenerative disorder (e.g., a psychosis secondary to Parkinson's disease, Alzheimer's disease, vascular dementia, or Lewy body dementia).

54. The method of any one of claims 48-52, wherein the disorder is schizophrenia or major depressive disorder.

55. The method of any one of claims 48-52, wherein the disorder is a negative symptom of schizophrenia.

56. The method of any one of claims 48-55, wherein the method comprises administering to the patient about 5 mg to about 45 mg of pimavanserin.

57. The method of any one of claims 48-56, wherein the method comprises administering to the patient about 34 mg of pimavanserin.

58. The method of any one of claims 48-56, wherein the method comprises administering to the patient about 20 mg of pimavanserin.

59. The method of any one of claims 48-56, wherein the method comprises administering to the patient about 17 mg of pimavanserin.

60. The method of any one of claims 48-56, wherein the method comprises administering to the patient about 10 mg of pimavanserin.

61. The method of any one of claims 48-60, wherein the enteral feeding tube is a nasogastric tube.

62. The method of any one of claims 48-60, wherein the enteral feeding tube is an orogastric tube.

63. The method of any one of claims 48-60, wherein the enteral feeding tube is a nasoenteric tube.

64. The method of any one of claims 48-60, wherein the enteral feeding tube is an oroenteric tube.

65. The method of any one of claims 48-64, wherein pimavanserin is dissolved in or mixed with about 5 mL to about 100 mL of the liquid vehicle.

66. The method of any one of claims 48-65, wherein pimavanserin is dissolved in or mixed with about 5 mL to about 60 mL of the liquid vehicle.

67. The method of any one of claims 48-66, wherein pimavanserin is dissolved in or mixed with about 15 mL of the liquid vehicle.

68. The method of any one of claims 48-66, wherein pimavanserin is dissolved in or mixed with about 30 mL of the liquid vehicle.

69. The method of any one of claims 48-68, wherein the administration further comprises the step of rising the nasogastric tube with an additional 5 mL to 20 mL of the liquid vehicle.

70. The method of any one of claims 48-69, wherein the contents of the capsule are mixed with about 5 mL to about 15 mL of the liquid vehicle or the contents of the capsule are sprinkled onto about 5 mL to about 15 mL of the soft food vehicle.

71. The method of any one of claims 48-70, wherein the ambient temperature is USP controlled room temperature or from 20° C. to 25° C.

72. The method of any one of claims 48-71, wherein the cold temperature is USP controlled cold temperature or from 2° C. to 8° C.

73. The method of any one of claims 48-72, wherein the patient has swallowing difficulties.

74. A method of treating a disorder in a patient in need thereof, the method comprising administering to the patient about 2 mg to about 80 mg of pimavanserin via a feeding tube once daily, wherein pimavanserin is dissolved in or mixed with about 5 mL to about 150 mL of a liquid vehicle before the administration.

75. The method of claim 74, wherein the disorder is a psychosis secondary to a neurodegenerative disorder (e.g., a psychosis secondary to Parkinson's disease, Alzheimer's disease, vascular dementia, or Lewy body dementia).

76. The method of claim 74, wherein the disorder is schizophrenia or major depressive disorder.

77. The method of claim 74, wherein the disorder is a negative symptom of schizophrenia.

78. The method of any one of claims 74-77, wherein the feeding tube is an oral feeding tube.

79. The method of any one of claims 74-77, wherein the feeding tube is an enteral feeding tube.

80. The method of any one of claims 74-79, wherein the method comprises administering to the patient about 5 mg to about 45 mg of pimavanserin.

81. The method of any one of claims 74-80, wherein the method comprises administering to the patient about 34 mg of pimavanserin.

82. The method of any one of claims 74-80, wherein the method comprises administering to the patient about 20 mg of pimavanserin.

83. The method of any one of claims 74-80, wherein the method comprises administering to the patient about 17 mg of pimavanserin.

84. The method of any one of claims 74-80, wherein the method comprises administering to the patient about 10 mg of pimavanserin wherein the patient is being treated concurrently with a strong CYP3A4 inhibitor.

85. The method of any one of claims 74-84, wherein the enteral feeding tube is an oroenteric tube.

86. The method of any one of claims 74-85, wherein pimavanserin is dissolved in or mixed with about 5 mL to about 100 mL of the liquid vehicle.

87. The method of any one of claims 74-86, wherein pimavanserin is dissolved in or mixed with about 5 mL to about 60 mL of the liquid vehicle.

88. The method of any one of claims 74-87, wherein pimavanserin is dissolved in or mixed with about 15 mL of the liquid vehicle.

89. The method of any one of claims 74-87, wherein pimavanserin is dissolved in or mixed with about 30 mL of the liquid vehicle.

90. The method of any one of claims 74-89, wherein the feeding tube is rinsed with an additional 5 mL to 20 mL of the liquid vehicle.

91. The method of any one of claims 74-90, wherein the patient has swallowing difficulties.

92. The method of any one of claims 1-91, wherein about 2 mg to about 80 mg of pimavanserin is a unit dose comprising pimavanserin tartrate.

93. The method of any one of claims 1-92, wherein about 4 mg to about 45 mg of pimavanserin is a unit dose comprising pimavanserin tartrate.

94. The method of any one of claims 1-93, wherein about 34 mg of pimavanserin is a unit dose comprising pimavanserin tartrate.

95. The method of any one of claims 1-93, wherein about 20 mg of pimavanserin is a unit dose comprising pimavanserin tartrate.

96. The method of any one of claims 1-93, wherein about 17 mg of pimavanserin is a unit dose comprising pimavanserin tartrate.

97. The method of any one of claims 1-93, wherein about 10 mg of pimavanserin is a unit dose comprising pimavanserin tartrate, wherein the patient is being treated concurrently with a strong CYP3A4 inhibitor.

98. The method of any one of claims 1-97, wherein the psychosis is Parkinson's disease psychosis or Alzheimer's disease psychosis.

99. The method of any one of claims 1-98, wherein the liquid vehicle is selected from the group consisting of water, applesauce, butter milk, coconut milk, cranberry juice, grapefruit juice, honey, maple syrup, milk, orange juice, pineapple juice, soybean milk, and vanilla shake.

100. The method of claim 99, wherein the liquid vehicle is selected from the group consisting of water, apple juice, applesauce, orange juice, and vanilla shake.

101. The method of claim 99 or 100, wherein the liquid vehicle is water.

102. The method of claim 99 or 100, wherein the liquid vehicle is applesauce or vanilla shake.

103. The method of any one of claims 48-98, wherein the soft food vehicle is selected from the group consisting of apples (puree), applesauce, orange juice, bananas (puree), carrots (puree), chocolate pudding, fruit jellies, fruit jam, orange marmalade, a nut or seed butter (such as peanut butter, sunflower seed butter or almond butter), rice pudding, strawberries (puree), strawberry jam, vanilla shake, and yogurt.

104. The method of claim 103, wherein the soft food vehicle is selected from the group consisting of chocolate pudding, a nut or seed butter (such as peanut butter, sunflower seed butter or almond butter), and yogurt.

105. The method of claim 103, wherein the soft food vehicle is selected from the group consisting of applesauce, orange juice, and vanilla shake.

106. The method of claim 103, wherein the soft food vehicle is selected from the group consisting of apples (puree), applesauce, bananas (puree), carrots (puree), chocolate pudding, fruit jellies, fruit jam, orange marmalade, a nut or seed butter (such as peanut butter, sunflower seed butter or almond butter), rice pudding, strawberries (puree), strawberry jam, vanilla shake, and yogurt.

107. The method of claim 103, wherein the soft food vehicle is selected from the group consisting of applesauce, chocolate pudding, vanilla shake, and yogurt.

108. A method of treating a disorder in a patient in need of pimavanserin, the method comprising administering to the patient 34 mg of pimavanserin once daily, wherein pimavanserin is dissolved in or mixed with about 5 mL to about 150 mL of a liquid vehicle at ambient or cold temperature before the administration.

109. A method of treating a disorder in a patient in need of pimavanserin, the method comprising administering to the patient 10 mg of pimavanserin once daily, wherein pimavanserin is dissolved in or mixed with about 5 mL to about 150 mL of a liquid vehicle at ambient or cold temperature before the administration, wherein the patient is being treated concurrently with a strong CYP3A4 inhibitor.

110. A method of treating a disorder in a patient in need of pimavanserin, the method comprising administering to the patient 34 mg of pimavanserin via an enteral feeding tube once daily, wherein pimavanserin is dissolved in or mixed with a liquid vehicle at ambient or cold temperature before the administration, wherein the solution or mixture is stable in the enteral feeding tube at ambient or cold temperature.

111. A method of treating a disorder in a patient in need of pimavanserin, the method comprising administering to the patient 10 mg of pimavanserin via an enteral feeding tube once daily, wherein pimavanserin is dissolved in or mixed with a liquid vehicle at ambient or cold temperature before the administration, wherein the solution or mixture is stable in the enteral feeding tube at ambient or cold temperature, wherein the patient is being treated concurrently with a strong CYP3A4 inhibitor.

112. A method of treating a disorder in a patient in need of pimavanserin, the method comprising administering to the patient 34 mg of pimavanserin once daily, wherein the administration comprises the steps of:
a) opening a pharmaceutically acceptable capsule comprising pimavanserin;
b) mixing the contents of the capsule with about 5 mL to about 150 mL of a liquid vehicle or sprinkling the contents of the capsule onto about 5 mL to about 150 mL of a soft food vehicle at ambient or cold temperature; and
c) delivering to the patient, optionally via an enteral feeding tube, the contents of the capsule with the liquid vehicle to the patient, or orally delivering the contents of the capsule with the soft food vehicle to the patient.

113. A method of treating a disorder in a patient in need of pimavanserin, the method comprising administering to the patient 34 mg of pimavanserin once daily, wherein the administration comprises the steps of:
a) opening a pharmaceutically acceptable capsule comprising pimavanserin;
b) mixing the contents of the capsule with about 5 mL to about 150 mL of a liquid vehicle or sprinkling the contents of the capsule onto about 5 mL to about 150 mL of a soft food vehicle at ambient or cold temperature; and
c) orally delivering the contents of the capsule with the soft food vehicle to the patient.

114. A method of treating a disorder in a patient in need of pimavanserin, the method comprising administering to the patient about 10 mg of pimavanserin once daily, wherein the administration comprises the steps of:
a) crushing a pharmaceutically acceptable tablet comprising pimavanserin;
b) mixing the crushed tablet with about 5 mL to about 150 mL of a liquid vehicle or sprinkling the crushed tablet onto about 5 mL to about 150 mL of a soft food vehicle at ambient or cold temperature; and
c) delivering to the patient, optionally via an enteral feeding tube, the crushed tablet with the liquid vehicle to the patient, or orally delivering the crushed tablet with the soft food vehicle to the patient;
wherein the patient is being treated concurrently with a strong CYP3A4 inhibitor.

115. A method of treating a disorder in a patient in need of pimavanserin, the method comprising administering to the patient about 10 mg of pimavanserin once daily, wherein the administration comprises the steps of:
a) crushing a pharmaceutically acceptable tablet comprising pimavanserin;
b) mixing the crushed tablet with about 5 mL to about 150 mL of a liquid vehicle or sprinkling the crushed tablet onto about 5 mL to about 150 mL of a soft food vehicle at ambient or cold temperature; and
c) orally delivering the crushed tablet with the soft food vehicle to the patient;
wherein the patient is being treated concurrently with a strong CYP3A4 inhibitor.

116. The method of any one of claims 108-115, wherein the disorder is a psychosis secondary to a neurodegenerative disorder.

117. The method of any one of claims 108-115, wherein the pimavanserin is in the form of the tartrate salt.

118. The method of claim 116, wherein the disorder is a psychosis secondary to Parkinson's disease, Alzheimer's disease, vascular dementia, or Lewy body dementia.

119. The method of any one of claims 108-115, wherein the disorder is schizophrenia or major depressive disorder.

120. A method of treating hallucinations and delusions associated with Parkinson's disease in a patient in need thereof, comprising administering to the patient a pimavanserin composition via a nasogastric tube once daily, wherein the pimavanserin composition comprises about 34 mg pimavanserin and about 5 mL to about 60 mL of a liquid vehicle.

121. A method of treating hallucinations and delusions associated with dementia in a patient in need thereof, comprising administering to the patient a pimavanserin composition via a nasogastric tube once daily, wherein the pimavanserin composition comprises about 34 mg pimavanserin and about 5 mL to about 60 mL of a liquid vehicle.

122. A method of treating hallucinations and delusions associated with dementia related to psychosis in a patient in need thereof, comprising administering to the patient a pimavanserin composition via a nasogastric tube once daily, wherein the pimavanserin composition comprises about 34 mg pimavanserin and about 5 mL to about 60 mL of a liquid vehicle.

123. A method of treating hallucinations and delusions associated with Parkinson's disease in a patient in need thereof, the method comprising administering to the patient a pimavanserin composition via a nasogastric tube once daily, wherein the pimavanserin composition comprises about 34 mg pimavanserin and a liquid vehicle, wherein the composition is stable in the nasogastric tube at ambient or cold temperature.

124. A method of treating hallucinations and delusions associated with Parkinson's disease in a patient in need thereof, comprising administering to the patient about 34 mg pimavanserin once daily, wherein the administration comprises the steps of:
a) opening a pharmaceutically acceptable capsule comprising pimavanserin;
b) mixing the contents of the capsule with about 5 mL to about 60 mL of a liquid vehicle or sprinkling the contents of the capsule onto about 5 mL to about 60 mL of a soft food vehicle at ambient or cold temperature; and
c) delivering via a nasogastric tube the contents of the capsule with the liquid vehicle to the patient, or orally delivering the contents of the capsule with the soft food vehicle to the patient.

125. A method of treating hallucinations and delusions associated with Parkinson's disease in a patient in need thereof, the method comprising administering to the patient a pimavanserin composition via an oral or enteral feeding tube once daily, wherein the pimavanserin composition comprises about 34 mg pimavanserin and about 5 mL to about 60 mL of a liquid vehicle.

126. A method of treating hallucinations and delusions associated with Parkinson's disease in a patient in need thereof, comprising administering to the patient a pimavanserin composition via a nasogastric tube once daily, wherein the pimavanserin composition comprises about 10 mg pimavanserin and about 5 mL to about 60 mL of a liquid vehicle, wherein the patient is being treated concurrently with a strong CYP3A4 inhibitor.

127. A method of treating hallucinations and delusions associated with dementia in a patient in need thereof, comprising administering to the patient a pimavanserin composition via a nasogastric tube once daily, wherein the pimavanserin composition comprises about 10 mg pimavanserin and about 5 mL to about 60 mL of a liquid vehicle, wherein the patient is being treated concurrently with a strong CYP3A4 inhibitor.

128. A method of treating hallucinations and delusions associated with dementia related to psychosis in a patient in need thereof, comprising administering to the patient a pimavanserin composition via a nasogastric tube once daily, wherein the pimavanserin composition comprises about 10 mg pimavanserin and about 5 mL to about 60 mL of a liquid vehicle, wherein the patient is being treated concurrently with a strong CYP3A4 inhibitor.

129. A method of treating hallucinations and delusions associated with Parkinson's disease in a patient in need thereof, the method comprising administering to the patient a pimavanserin composition via a nasogastric tube once daily, wherein the pimavanserin composition comprises about 10 mg pimavanserin and a liquid vehicle, wherein the composition is stable in the nasogastric tube at ambient or cold temperature, wherein the patient is being treated concurrently with a strong CYP3A4 inhibitor.

130. A method of treating hallucinations and delusions associated with Parkinson's disease in a patient in need thereof, comprising administering to the patient about 10 mg pimavanserin once daily, wherein the administration comprises the steps of:
a) opening a pharmaceutically acceptable capsule comprising pimavanserin;
b) mixing the contents of the capsule with about 5 mL to about 60 mL of a liquid vehicle or sprinkling the contents of the capsule onto about 5 mL to about 60 mL of a soft food vehicle at ambient or cold temperature; and
c) delivering via a nasogastric tube the contents of the capsule with the liquid vehicle to the patient, or orally delivering the contents of the capsule with the soft food vehicle to the patient;
wherein the patient is being treated concurrently with a strong CYP3A4 inhibitor.

131. A method of treating hallucinations and delusions associated with Parkinson's disease in a patient in need thereof, the method comprising administering to the patient a pimavanserin composition via an oral or enteral feeding tube once daily, wherein the pimavanserin composition comprises about 10 mg pimavanserin and about 5 mL to about 60 mL of a liquid vehicle, wherein the patient is being treated concurrently with a strong CYP3A4 inhibitor.

132. The method of any one of claims 120-131, wherein the patient has swallowing difficulties.
